(19) United States
(12) Patent Application Publication
(10) Pub. No.: US 2024/0393328 A1
(43) Pub. Date: Nov. 28, 2024

(54) Wireless Power-up and Readout of Label-free Electronic Detection of Protein Biomarkers

(71) Applicant: RUTGERS, THE STATE UNIVERSITY OF NEW JERSEY, New Brunswick, NJ (US)

(72) Inventors: Hassan Raji, Piscataway, NJ (US); Mehdi Javanmard, Princeton Junction, NJ (US); Pengfei Xie, Piscataway, NJ (US); Seyed Reza Mahmoodi, Denver, CO (US)

(21) Appl. No.: 18/694,664

(22) PCT Filed: Sep. 30, 2022
(86) PCT No.: PCT/US2022/077403

§ 371 Date: Mar. 22, 2024

Related U.S. Application Data
(60) Provisional application No. 63/261,995, filed on Oct. 1, 2021.

(51) Int. Cl. G01N 33/543; B01L 3/00

(52) U.S. Cl. CPC G01N 33/5438; B01L 3/502761; B01L 2300/0645; B01L 2300/0893

(57) ABSTRACT

A device for detecting a target analyte in a sample includes a nanowell impedance sensor configured to receive a sample including a target analyte. An impedance between electrodes of the sensor is modulated by the target analyte when present in the sample, the modulated impedance being indicative of a concentration of the target analyte in the sample. A receiver circuit, connected across the electrodes, has a receiver resonance frequency and includes an inductive coil to induce an electrical current in the receiver circuit while inductively coupled with a physically separate transmitter circuit having a transmitter resonance frequency. The receiver resonance frequency is within a resonance overlap percentage of the transmitter resonance frequency. The electrical current induced in the receiver circuit is a time-varying signal having a frequency within a signal overlap percentage of the receiver resonance frequency. The modulated impedance is determined as a function of the electrical current induced, thereby detecting the presence of the target analyte, and the concentration thereof, in the sample.

### RELATED APPLICATION

This application claims the benefit of U.S. Provisional Application No. 63/261,995, filed on Oct. 1, 2021. The entire teachings of the above application(s) are incorporated herein by reference.

### GOVERNMENT SUPPORT

This invention was made with government support under Grant Number W911NF-20-1-0295 awarded by Defense Advanced Research Project Agency (DARPA) and the Army Research Office (ARO). The government has certain rights in the invention.

### BACKGROUND

Wireless power transfer systems are an emerging technology that may be used to power various portable and biomedical devices [1]. Meanwhile, label-free biosensing technologies have shown considerable promise to facilitate protein quantification in clinical settings. Construction of a nanowell impedance sensor has been demonstrated [2]. Such a sensor monitors changes in impedance across electrodes of the nanowell impedance sensor in response to target analytes such as proteins binding within or becoming unbound from within wells of the nanowell impedance sensor.

### SUMMARY

A device for detecting a target analyte in a sample includes a nanowell impedance sensor and a receiver circuit. The nanowell sensor is configured to receive a sample including a target analyte, an impedance between electrodes of the sensor being modulated by the target analyte when the target analyte is present in the sample, the modulated impedance being indicative of a concentration of the target analyte in the sample.

The term “analyte” as used herein means a protein, peptide, and more specifically a cytokine (e.g., TNF-α, IL-1, IL-4, IL-6, IL-8, IL-10, IL-13) and other biomarkers (e.g., tumor biomarkers and other proteins known to link to diseases), hormones (e.g., insulin, cortisol), nucleic acids, toxins, small molecules, and any substances that can bind to a probe molecule. Cytokines are a broad category of small proteins (˜5-20 kDa) that are important in cell signaling.

The nanowell impedance sensor is an embodiment of a sensor as described in U.S. Patent Publication No. US 2020/0261907, which is being incorporated by reference herein.

The receiver circuit is connected across the electrodes and has a receiver resonance frequency. The receiver circuit includes an inductive coil configured to induce an electrical current in the receiver circuit while inductively coupled with a physically separate transmitter circuit having a transmitter resonance frequency, the receiver resonance frequency being within a resonance overlap percentage of the transmitter resonance frequency. The electrical current induced in the receiver circuit is a time-varying signal having a frequency within a signal overlap percentage of the receiver resonance frequency. The modulated impedance is determinable as a function of the electrical current induced, thereby detecting the presence of the target analyte, and the concentration thereof, in the sample.

The inductive coil of the receiver circuit has multiple turns, which can be characterized by a distance between turns and a turn width, to be in a suitable range for a particular application. For example, the inductive coil can have a number of turns between 5 and 60, a distance between turns of between 10 and 30 microns, and/or a turn width between 10 and 30 microns.

The inductive coil of the receiver circuit can be characterized by, for example, a square or a circular spiral geometry.

The receiver circuit can include at least one inductor connected in series with the inductive coil of the receiver circuit, the at least one inductor enabling the receiver resonance frequency to remain within the resonance overlap percentage of the transmitter resonance frequency.

For example, the resonance overlap percentage can be 10%. The signal overlap percentage can be at least 60%. The resonance frequency of the receiver can be within a range of 4 to 50 MHz.

The device can occupy an area of less than one square centimeter, so as to be implantable within a human or animal body or a part thereof.

A measurement system for detecting a target analyte in a sample includes a nanowell impedance sensor, a receiver circuit, and a transmitter circuit. The nanowell impedance sensor is configured to receive a sample including a target analyte, an impedance between electrodes of the sensor being modulated by the target analyte when the target analyte is present in the sample, the modulated impedance being indicative of a concentration of the target analyte in the sample. The receiver circuit is connected across the electrodes of the sensor, includes an inductive coil, and has a receiver resonance frequency. The transmitter circuit has a transmitter resonance frequency such that the receiver resonance frequency is within a resonance overlap percentage of the transmitter resonance frequency. The transmitter circuit includes a voltage source configured to apply an electrical voltage to the inductive coil of the transmitter circuit, so as to induce the electrical current in the receiver circuit, causing an electrical voltage to be applied across the electrodes of the nanowell sensor. An inductive coil of the transmitter circuit is configured to be wirelessly coupled with the inductive coil of the receiver circuit by mutual inductance such that the receiver circuit is inductively coupled with the transmitter circuit. The electrical current induced in the receiver circuit is a time-varying signal having a frequency within a signal overlap percentage of the receiver resonance frequency. The modulated impedance is determined as a function of the electrical current induced, thereby detecting the presence of the target analyte, and the concentration thereof, in the sample.

The transmitter circuit can include a lock-in amplifier and a measurement device configured to measure a current flowing in the transmitter circuit to enable the modulated impedance to be determined further as a function of a current measured.

The transmitter circuit can include a measurement device configured to measure an impedance spectrum across the inductive coil of the transmitter circuit to enable the modulated impedance to be determined further as a function of a measured change in the resonance frequency of the receiver circuit in response to a change in the concentration of the target analyte present in the sample.

The receiver circuit can include at least one inductor connected in series with the inductive coil of the receiver circuit, the at least one inductor enabling the receiver resonance frequency to remain within the resonance overlap percentage of the transmitter resonance frequency.

The system can be characterized by a resonance overlap percentage and a signal overlap percentage. For example, the resonance overlap percentage can be 10%. The signal overlap percentage can be at least 60%.

The receiver resonance frequency and the transmitter resonance frequency can be within a range of 4 to 50 MHz.

In the measurement system, the inductive coil of the transmitter circuit can have a number of turns between 10 and 200.

A method of manufacturing a measurement system for detecting a target analyte in a sample includes determining an appropriate number of turns, turn thickness, turn shape, and spacing between turns for an inductive coil to be included in a receiver circuit, and an appropriate number of turns and turn diameter for an inductive coil to be included in a transmitter circuit that is physically separate from the receiver circuit, to establish a receiver resonance frequency of the receiver circuit and a transmitter resonance frequency of the transmitter circuit such that the receiver resonance frequency is within a resonance overlap percentage of the transmitter resonance frequency while the receiver circuit is inductively coupled with the transmitter circuit.

The method can further include determining an appropriate inductance value for at least one inductor to be electrically connected in series with the inductive coil of the receiver circuit, the at least one inductor enabling the receiver resonance frequency to remain within the resonance overlap percentage of the transmitter resonance frequency.

In one example, the resonance overlap percentage is 10% and the signal overlap percentage is at least 60%.

The method can include selecting a turn shape and values for number of turns, turn thickness, and spacing between turns for the inductive coil of the receiver circuit to establish the receiver resonance frequency within a range of 4 to 50 MHz. The method can further include selecting a number of turns and turn diameter for the inductive coil of the transmitter circuit to establish the transmitter resonance frequency within a range of 4 to 50 MHz.

The method can include lithographically fabricating the inductive coil of the receiver circuit as a planar coil disposed upon an implantable substrate.

The method can include electrically connecting a lithographically fabricated inductive coil of the receiver circuit to electrodes of a nanowell sensor that is (i) co-fabricated with the inductive coil upon an implantable substrate, wherein the implantable substrate, after singulation from a parent wafer, has no dimension greater than one centimeter so as to facilitate implantation within a human or animal body or a part thereof; or (ii) physically mated with the implantable substrate within an implantable module, wherein the implantable module has no dimension greater than one centimeter so as to facilitate implantation within a human or animal body or a part thereof.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a schematic diagram of an example system for detecting target analytes in a sample according to embodiments of the present disclosure.

FIG. 1B is a schematic diagram of an example nanowell array used in the system of FIG. 1A.

FIG. 1C is a schematic diagram of an example nanowell used in the nanowell array of FIG. 1B.

FIGS. 2A-2B are example plots of output voltage of a nanowell sensor employed in embodiments according to the present disclosure and undergoing changes to various environmental conditions.

FIG. 3 is a schematic diagram of an example embodiment of receiver and transmitter circuits of a system for detecting target analytes.

FIG. 4A is a line drawing depicting an example system used to detect target analytes wherein multiple components are used for frequency tuning.

FIG. 4B is a line drawing depicting components used for frequency tuning in the system of FIG. 4A.

FIG. 5 is a line drawing depicting an example system used to detect target analytes wherein a single inductor is used for frequency tuning.

FIGS. 6A-6D are example spectral plots showing effects of various aspects of inductive coil design in the system of FIG. 5.

FIG. 7A is a schematic of an example inductive coil used in embodiments.

FIG. 7B is a line drawing showing a top view of the inductive coil of FIG. 7A.

FIG. 7C is a line drawing showing a bottom view of the inductive coil of FIG. 7A.

FIG. 7D is a line drawing showing a combined view of the inductive coil of FIG. 7A with another inductive coil inductively coupled therewith.

FIGS. 8A-8F are example plots showing data for the example inductive coil of FIG. 7A.

FIGS. 9A and 9B are example spectral plots for inductive coils such as those shown in FIG. 7D.

FIGS. 9C and 9D are example plots showing data for inductive coils such as those shown in FIG. 7D.

FIGS. 10A and 10B are example spectral plots for inductive coils such as those shown in FIG. 7D.

FIGS. 10C and 10D are example plots showing data for inductive coils such as those shown in FIG. 7D.

FIGS. 11A-D are example spectral plots demonstrating frequency shifts for inductive coils such as those shown in FIG. 7D.

FIGS. 12A and 12B are line drawings showing components of a microfluidic test bed according to an example embodiment.

FIGS. 13 and 14 are diagrams depicting example embodiments of a method of manufacturing a measurement system for detecting a target analyte in a sample according to embodiments of the present disclosure.

FIGS. 15 and 16 are schematic diagrams of example embodiments of receiver and transmitter circuits of a system for detecting target analytes.

FIG. 17 is a schematic of example inductive coils used in embodiments.

### DETAILED DESCRIPTION

A description of example embodiments follows.

Electrical and electrochemical sensing provide a valuable platform for immunosensing technologies. Biosensors are involved in various applications of electrical and electrochemical sensing, including medical diagnostics and healthcare monitoring. Additionally, wireless power transfer is suited for remote use of biosensors because no direct electrical connection is required. Embodiments of the present disclosure provide methods for wireless readout of a label-free electronic biosensor for the detection of target analytes using inductive coupling. Such analytes may include, for example, proteins or other biomarkers.

Some embodiments provide a sensing platform for detecting protein biomarkers using wireless power-up and read-outs. In some embodiments, such a sensing platform can be applied as biosensing implants. Some embodiments provide a wireless biosensing platform using resonant inductive coupling, including features designed for transducing small impedance changes during biosensing by the nanowell sensor arrays. The geometry of the primary coil is specifically designed so that it can be used with the nanowell sensor in a specific frequency range (for example, around 10 MHZ). Some embodiments include a lock-in amplifier. Based on a primary coil resonance frequency, a secondary coil may be designed in such a way that a resonance frequency thereof aligns with the resonance frequency of the primary coil. Design considerations of such a secondary coil (also referred to as a microcoil) may include coil geometry and aspects thereof. In some embodiments, at least one of the primary and secondary coils are produced by microfabrication. Some embodiments are able to measure very small changes in impedance (for example, less than 0.1 kΩ).

Implantable medical devices are useful for continuous monitoring and early detection of pathological conditions. Transmitting signals from the implanted devices can be a major challenge since wiring in intradermal and subcutaneous locations is inconvenient.

Some embodiments are related to a method for transmitting changes in an impedance signal due to binding of protein biomarkers in a nanowell sensor array. The electrical signal detected is captured via resonance inductive coupling. A prototype has been built and tested using phosphate-buffered saline (PBS) spiked with high mobility group box protein 1 (HMGB1).

Some embodiments are related to a system that provides a sensing platform for detecting target analytes in a sample using wireless power-up and read-out. In some embodiments, the system includes a power transmitter and a receiver coil for electromagnetically coupled wireless power transfer. In these embodiments, there is no direct electrical connection between the receiver and transmitter side.

In some embodiments, the receiver coil is connected to a biosensor that consists of two overlapping electrodes that measure impedance, and there is a thin oxide layer between them. The electrodes may be in an array format, and antibody immobilization may be probed inside several nanometer-sized wells. Embodiments such as this inductively coupled platform can be applied to a wide array of protein biomarkers.

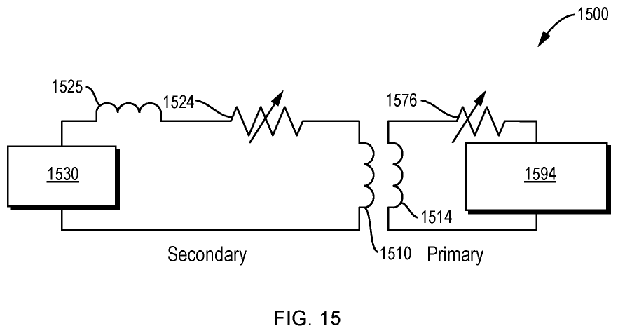

A nanowell impedance sensor monitors the impedance across the electrodes and responds to the target analyte bindings. An impedance change due to protein immobilization to antibodies in the receiver side is translated into a change in the output voltage of a lock-in-amplifier in the transmitter side. In some embodiments, an electrochemical impedance spectroscopy (EIS) measurement is recorded from the transmitter at a resonance frequency of the transmitter circuit. A variable component, such as a variable resistor or inductor, can be added to either the transmitter or receiver side, or both, to fine tune the adjustment of resonance frequency. Such variable components are described further herein with reference to FIG. 15.

The resonance frequency of the transmitter circuit may be equal or approximately equal to the resonance frequency of the primary coil. Alternatively, the resonance frequency of the transmitter circuit may be adjusted based on the resonance frequency of the primary coil by including passive or active electronic components in the transmitter circuit. Such passive or active components may include, for example, resistors, capacitors, inductors, or amplifiers. A resonance frequency of the receiver circuit may be similarly adjusted based on the resonance frequency of the secondary coil by including passive or active components in the receiver circuit as described with respect to the transmitter circuit.

In some embodiments, a wireless power transfer (WPT) nanowell impedance sensor is employed to detect, for example, HMGB1, or another from a wide array of protein biomarkers. In these embodiments, a process of protein immobilization by antibodies may be translated into a change in an output voltage of a lock-in-amplifier on the transmitter side, which can be detected remotely from the sensor, i.e., at the transmitter side. Wireless power-up thus enables performance of measurements without a direct and physical connection between the sensor and the measurement tools. In embodiments designed for implantation within a human or animal body, or a part thereof, inclusion of wireless power-up features therefore represent a major step forward in the development and miniaturization of the system for implantation.

Nanowell impedance sensors, as introduced above, may herein be referred to interchangeably as “nanowell sensors” or simply as “sensors.” In some embodiments, a nanowell sensor [2] includes two electrodes, measuring impedance therebetween. A thin dielectric oxide layer may be present between the two electrodes. This design creates a conductive path between the two overlapping electrodes through a testing solution present in nanowells of the nanowell sensor. Real-time measurement of impedance may thus be carried out using the two electrodes. The electrodes are in an array format, which enables examination of antibody immobilization inside the nanowells.

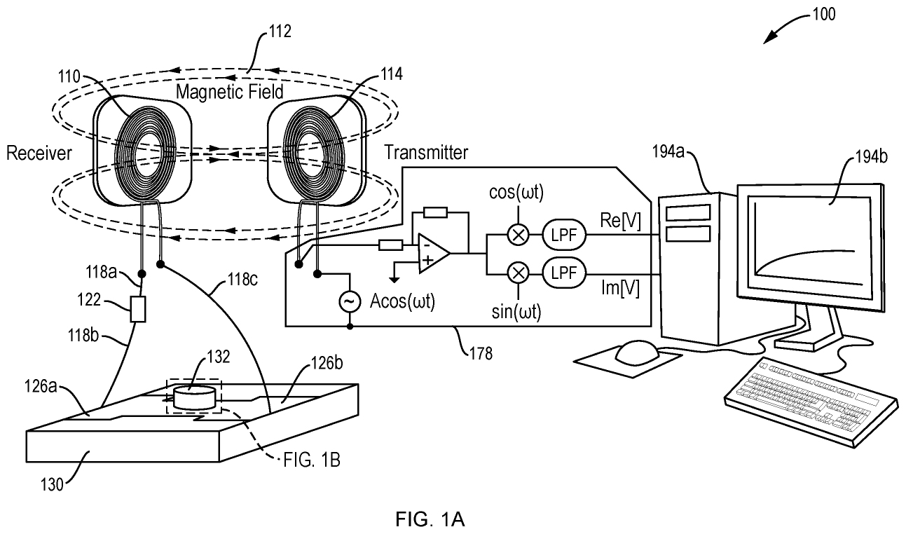

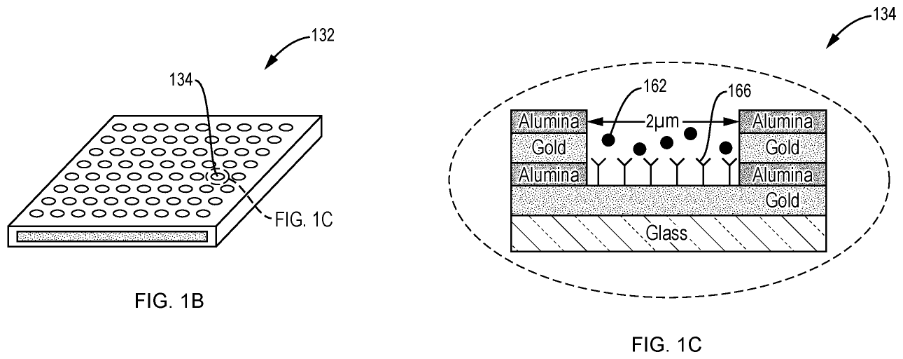

FIG. 1A shows a schematic diagram of an example embodiment of a system 100 for detecting a target analyte in a sample. In the embodiment, a measurement system 100 is a wireless protein biosensor system that includes two inductively coupled coils, i.e., a transmitter coil 114 and a receiver coil 110. A magnetic field 112 associated with said inductive coupling is shown. For convenience, the transmitter 114 and receiver 110 coils are illustrated in FIG. 1A as being similar in size and shape, but such similarity is not required. For example, the receiver coil 110 may be smaller than the transmitter coil 114. The receiver coil 110 can be a microcoil, such as is further described hereinbelow.

To continue, a nanowell sensor 130 may be included with the receiver coil 110 in a receiver circuit. The nanowell sensor 130 is configured to receive a sample including a target analyte. An electrode 126a of the nanowell sensor 130 is connected in series with the receiver coil 110 by wires 118a, 118b and at least one additional inductor 122. The additional inductor(s) 122 may be selected to modify the resonance frequency of the receiver circuit. Another electrode 126b of the nanowell sensor 130 is connected via wire 118c to the side of the receiver coil 110 that is opposite to the aforementioned electrode 126a. There is no direct electrical connection between the receiver and transmitter circuits. The nanowell sensor 130 includes an array of nanowells 132. When the impedance inside the nanowells 132 changes, due to, for example, analytes disposed therein connecting to binding sites at a rate proportional to the concentration of the analytes in the sample, this change in impedance can be monitored by a lock-in amplifier measurement system 178 in the transmitter circuit. The lock-in amplifier measurement system 178 may be configured to determine net complex impedance. For example, the lock-in amplifier measurement system 178 may be configured to measure a current flowing in the transmitter circuit to enable the modulated impedance to be determined further as a function of a current measured. Measurement results may be displayed on a computer 194a display 194b associated with the lock-in amplifier measurement system 178. The receiver circuit may comprise a plurality of sensor devices 130. Each sensor device 130 of the plurality may include a receiver circuit as described above, and may be configured to be inductively coupled to the transmitter circuit. An example of a system including two sensor devices and a transmitter circuit (e.g. primary coil) is described hereinbelow.

The transmitter circuit includes a voltage source, e.g., as part of the lock-in amplifier measurement system 178. Such a voltage source may be configured to apply an electrical voltage to the primary inductive coil 114 of the transmitter circuit so as to induce the electrical current in the secondary inductive coil 110 of the receiver circuit, causing an electrical voltage to be applied across the electrodes 126a, 126b of the nanowell sensor 130. The primary inductive coil of the transmitter circuit 114 is thus configured to be wirelessly coupled with the secondary inductive coil 110 of the receiver circuit by mutual inductance such that the receiver circuit is inductively coupled with the transmitter circuit. The electrical current induced in the receiver circuit is a time-varying signal having a frequency within a signal overlap percentage of the receiver resonance frequency. The modulated impedance is determined as a function of the electrical current induced, thereby detecting the presence of the target analyte, and the concentration thereof, in the sample.

The transmitter circuit can include a measurement device configured to measure an impedance spectrum across the inductive coil of the transmitter circuit to enable the modulated impedance to be determined further as a function of a measured change in the resonance frequency of the receiver circuit in response to a change in the concentration of the target analyte present in the sample.

FIG. 1B shows a schematic diagram of an example embodiment of a nanowell array 132, with individual nanowells 134 pictured therein. FIG. 1C shows a schematic diagram of an example embodiment of a nanowell 134, with analytes 162 disposed therein connecting to binding sites 166. Such connection of analytes 162 to binding sites 166 causes the impedance inside the nanowell array 132 to change as described above with respect to FIG. 1A.

An experiment may be performed, for example, in triplicate, to evaluate an extent to which target analytes 162, including proteins or other biomarkers, can be detected by the nanowell sensor 130 in configurations such as that of the aforementioned example embodiment. Reagents may be added to a round-shaped polydimethylsiloxane (PDMS) well with a diameter of, for example, 5 mm. The PDMS well may be disposed on top of an array of nanowells 132 within the nanowell sensor 130. The array of nanowells 130 may be fabricated in a similar way as described in [2]. Reagents may be manually added to the well, sequentially, for example, in six major steps.

In some embodiments, an inductively coupled nanowell impedance sensor 130 may be fabricated and implemented for label-free detection of target protein biomarkers. In such embodiments, an array of nanowells 132 within the sensor are functionalized with antibody, and embedded with electrodes 126a, 126b to track changes in ionic resistance within the nanowells 132. When there is a binding of a target protein or analyte 162 to the corresponding antibody or binding site 166 inside individual nanowells 134, an inductively coupled wireless power transfer system such as system 100 may translate changes in impedance observed by the sensor 130 to an equivalent impedance as seen from the transmitter circuit, as shown and described hereinabove.

The receiver circuit is connected across the electrodes 126a, 126b and has a receiver resonance frequency. The secondary coil 110 of the receiver circuit is an inductive coil configured to induce an electrical current in the receiver circuit while inductively coupled with, and physically separate from, the primary coil 114 of the transmitter circuit. The transmitter circuit has a transmitter resonance frequency, and the receiver resonance frequency is within a resonance overlap percentage of the transmitter resonance frequency. The electrical current induced in the receiver circuit is a time-varying signal having a frequency within a signal overlap percentage of the receiver resonance frequency. The modulated impedance is determinable as a function of the electrical current induced, thereby detecting the presence of the target analyte 162, and the concentration thereof, in the sample.

The secondary inductive coil 110 of the receiver circuit has multiple turns, which can be characterized by a distance between turns and a turn width, to be in a suitable range for a particular application. For example, the secondary inductive coil 110 can have a number of turns between 5 and 60, a distance between turns of between 10 and 30 microns, and/or a turn width between 10 and 30 microns.

The secondary inductive coil 110 of the receiver circuit can be characterized by, for example, a square or a circular spiral geometry. The receiver circuit can include at least one inductor 122 connected in series with the secondary inductive coil 110, the at least one inductor enabling the receiver resonance frequency to remain within the resonance overlap percentage of the transmitter resonance frequency. For example, the resonance overlap percentage can be 10%. The signal overlap percentage can be at least 60%. The resonance frequency of the receiver circuit can be within a range of 4 to 50 MHz. In the measurement system 100, the primary inductive coil 114 of the transmitter circuit can have a number of turns between 10 and 200.

A device for detecting a target analyte in a sample may include a measurement system such as the system 100. The device can occupy an area of less than one square centimeter, so as to be implantable within a human or animal body or a part thereof.

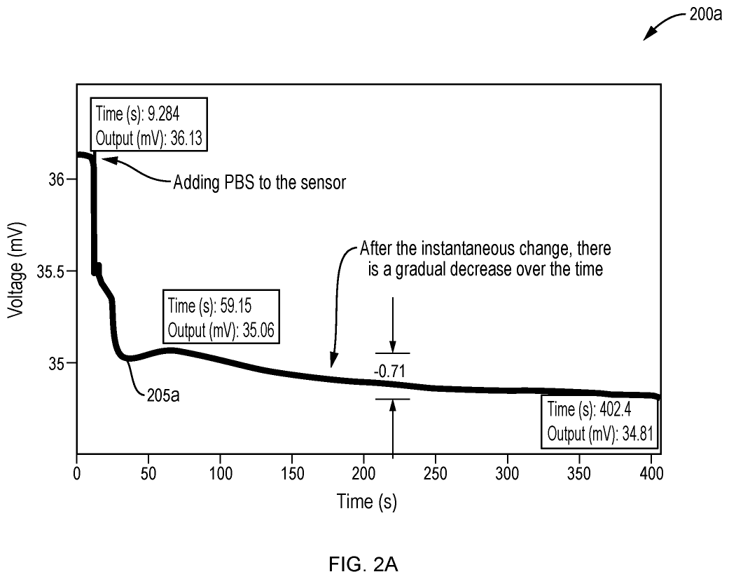

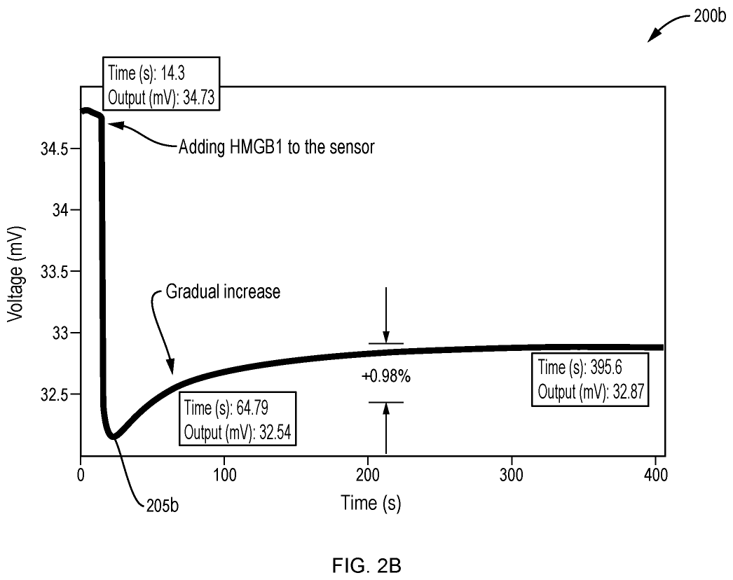

FIG. 2A shows an example plot 200a of experimental results for nanowell sensor output voltage 205a when the nanowell sensor 130 is subjected to a negative control. In the first step of an experiment performed as described above, the nanowell sensor 130 was filled with 10 μl blank PBS sample, before 1 μl of the same was added as a negative control (NC). A set of three iterations of the experiment showed an average instantaneous change in lock-in amplifier output voltage of −54.433% and −2.43% with a standard deviation (SD) of 3.37 and 1.42 respectively for blank PBS and NC.

Subsequently in the experiment, 5 μl of antibody was added to the nanowell sensor 130. After an instantaneous change in the baseline, there was a gradual and exponential increase in the output voltage. This is consistent with time-dependent behavior of antigen-antibody binding observed in a previous study using a wired nanowell sensor. In contrast with NC, a p-value of 0.00891 was thus observed. Then, the fluid inside the nanowell sensor 130 was taken out to remove extra antibodies, and 10 μl of PBS was added to the nanowell sensor 130 before another 1 μl of NC was added thereto.

FIG. 2B shows an example plot 200b of experimental results for nanowell sensor output voltage 205b when the nanowell sensor 130 is subjected to a protein sample. The experiment described hereinabove continued as 3 μl of protein solution was added to the nanowell sensor 130, leading to a gradual exponential increase in the output voltage 205b, similar to the what was observed in the aforementioned previous study with the wired nanowell sensor. The observed average change in output voltage 205b was 0.98% for protein in the positive direction with a standard deviation of 0.62. Yet, the observed average change for NC was 1.2% in the negative direction with a standard deviation of 0.43. A p-value between these respective sets of data for the three experiments with NC and protein was observed to be 0.00788 (<0.01).

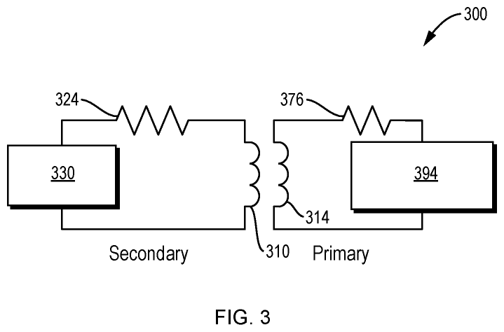

FIG. 3 is a schematic diagram 300 illustrating an example embodiment, wherein the transmitter circuit includes a resistor 376, a primary coil 314, and a lock-in-amplifier 394, e.g. a Zurich impedance spectroscope (Zurich Instruments HF2IS, Zurich, SI). In one example, the resistor 376 is a 500-ohm resistor. The lock-in amplifier 394 measures the complex impedance in real-time to detect binding events involving proteins or other biomarkers in the nanowell sensor 330. The primary coil 314 is inductively coupled with a secondary coil 310 connected in series with the nanowell sensor 330 and a resistor 324 in the receiver circuit. The resistor 324 may be, e.g., 26 ohms.

In embodiments similar to that which is depicted in FIG. 3, the primary coil 314 may be, e.g., a 40 turns coil with a diameter of, e.g., 2.4 cm. An alternating-current (AC) excitation source may be included in the transmitter circuit and set to, e.g., 400 mV at a measured resonance frequency of, e.g., 10.5 MHz. To measure impedance spectrum and phase in a wide range of frequency, an impedance analyzer (IA) may be employed, such as an E4990A IA, made by Keysight Technologies (CA, USA). Other IAs, or other coil geometries and AC excitation settings, may alternatively be used.

### Exemplification 1

Some embodiments of the measurement system 100 facilitate detection of target analytes such as antibodies and proteins using a microcoil as the secondary inductive coil 110, with a plurality of inductors 122 connected in series with the secondary coil 110. The inductors 122 are included to increase sensitivity of detection of target analytes by the measurement system 100. In such embodiments, initially, the resonance frequencies of primary 114 and secondary 110 coils may not overlap well. A constraint may be that a smaller secondary coil 110 results in a larger resonance frequency. In this respect, an effective resonance frequency of the secondary coil 110 may be changed by adding some components, such as the inductors 122, to make these two resonance frequencies overlap. Additionally, a Veroboard or other type of stripboard may be used instead of a breadboard while performing experiments to evaluate resonance frequency overlap. Moreover, length of wires may be reduced to improve resonance frequency overlap.

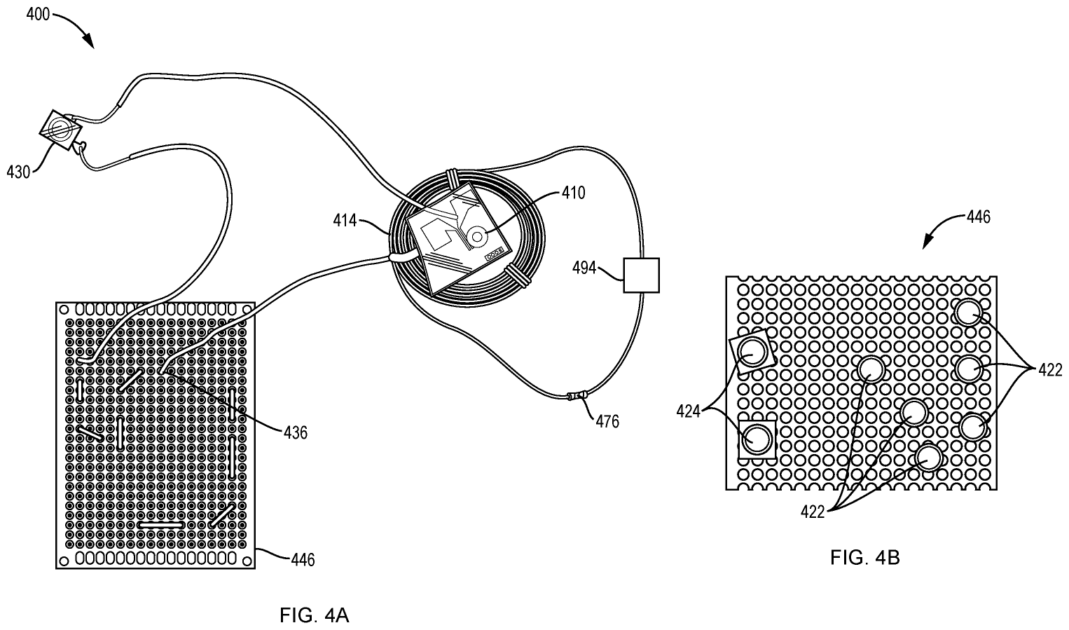

FIG. 4A is a line drawing depicting an example system 400 used to perform such experiments. In the example system 400, a nanowell impedance sensor 430 is in series with a microcoil 414 and components are soldered on a Veroboard 446. The secondary coil 414 may be referred to interchangeably herein as a “microcoil.” Also depicted in FIG. 4A are primary coil 410 and a lock-in amplifier measurement system, specifically, a Zurich impedance spectroscope 494. A resistor 476 is additionally shown to be connected in series with primary coil 410 and spectroscope 494.

FIG. 4B is a line drawing depicting a view of the Veroboard 446, on the opposite side of the Veroboard 446 from FIG. 4A. Components can be seen to be soldered on the Veroboard 446. Such components include inductors 422 and resistors 424.

Considering self-resonance frequency of the inductors 422, the resonance frequency of the secondary coil 410 may be adjusted in a way that the secondary resonance frequency becomes close to that of the primary coil 414. An impedance spectrum may then be measured. The number of turns on the primary coil 414 may be fixed.

An example method for determining an appropriate combination of components such as inductors 422 may be as follows. Initially, a spectrum of the primary coil 414 may be measured with the Zurich Impedance Spectroscope 494 to find the resonance frequency of the primary coil 414. Then, the components may be added in series and/or parallel with the secondary coil 410, and the resonance frequency of secondary circuit may be measured.

These steps may be repeated until the resonance frequencies of the secondary and primary circuits are close to one another.

In the example system 400, the resonance frequency of the primary coil 414 (7.93 MHz) was found to almost overlap the resonance frequency of the secondary coil 410 (8.01 MHz). A number of turns for the primary coil 414 may be, for example, 38 turns, as in the example system 400. Sensitivity for the example system 400 has been improved by more than 100× compared to results for a previous setup, wherein primary and secondary resonance peaks did not overlap.

Antibody detection experiments for the example system 400 were performed in triplicate. In a first step, by adding PBS to a dry sensor 430, a change in the output signal was observed. The change was −18.9, −10.9, −19.4 in the three respective trials. A dramatic change in the signal was thus observed. In a second step, to perform a negative control experiment for the example system 400, a blank PBS sample was added to a PDMS well of the sensor 430. The experiment was performed in triplicate. The output increased initially and then gradually over time. In a next step using the example system 400, an antibody sample was added to the PDMS well. This addition resulted in an instantaneous change and then a gradual decrease in the output over a period of time. The gradual increase in the negative control experiment could be clearly differentiated from the initial change and subsequent gradual decrease caused by the antibody absorption. The next step with the example system 400 was to remove the antibody sample from the PDMS well and to add a buffer, specifically PBS, to the PDMS well. This resulted an increase in output voltage for removal, and a decrease in output voltage for addition of the buffer. Finally, for the example system 400, by addition of an IL-6 sample to the PDMS well, there was an instantaneous change in the output signal and then a gradual decrease over time.

### Exemplification 2

In example system 400, multiple components were used in series/parallel with the microcoil 410 in order to produce an effective resonance frequency of the microcoil similar to the resonance frequency of the primary coil 414. To minimize the number of components in the secondary circuit, a design goal may be to use only one inductor soldered on a Veroboard. With example system 400, measurements were done in a way such that the parameters relevant to the primary coil 414 were fixed. Such fixed parameters of the primary coil 414 included a number of turns thereof.

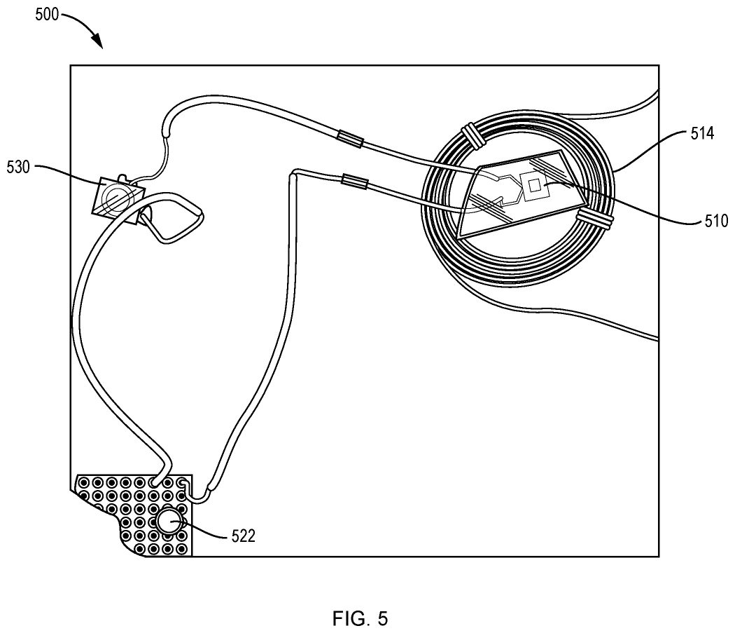

FIG. 5 is a line drawing depicting an example system 500 used to detect target analytes in a nanowell sensor 530, in which system 500 only a single inductor 522 is used in series with the secondary coil 510. The secondary coil 510 appears as a substantially square microcoil, positioned near the primary coil 514.

To use only one inductor in the secondary circuit, first, the spectrum of the secondary coil 510 may be measured while the secondary coil 510 is in series with an inductor 522 and the nanowell sensor 530 to find the resonance frequency of the secondary circuit. Then, the primary coil 514 may be wired with a specific diameter and number of turns. In this way, the resonance frequency of the primary coil 514 may be adjusted. Meanwhile, this resonance frequency of the primary coil 514 may be measured. Next, some of the turns of the primary coil 514 may be cut in such a way that the respective resonance frequencies of the secondary 510 and primary 514 coils become close to one another. Not only do the number of turns change this resonance, but also the relative position of the turns can affect it. Thus, the relative position may be kept constant while only the number of turns is changed (cutting, for example, 1 turn in each trial).

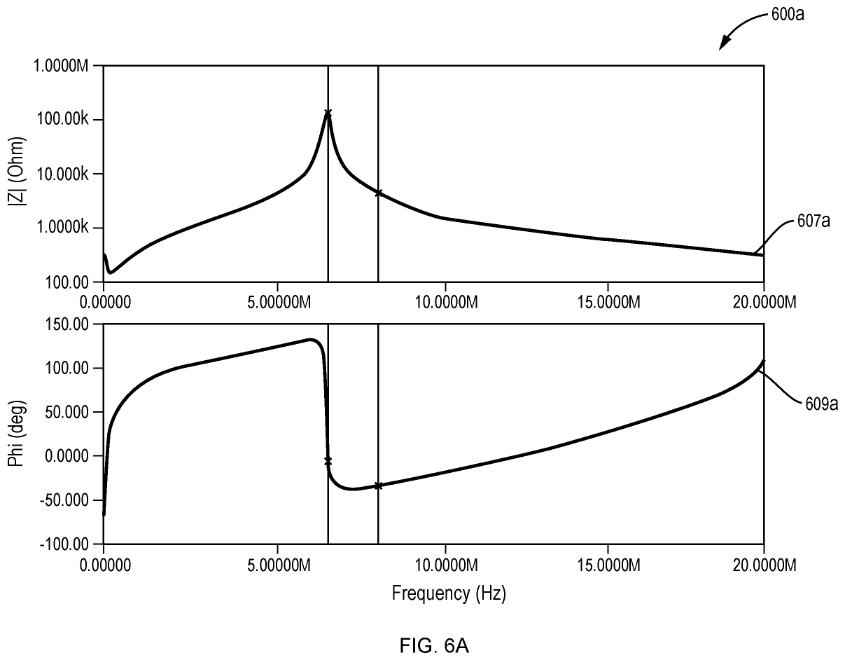

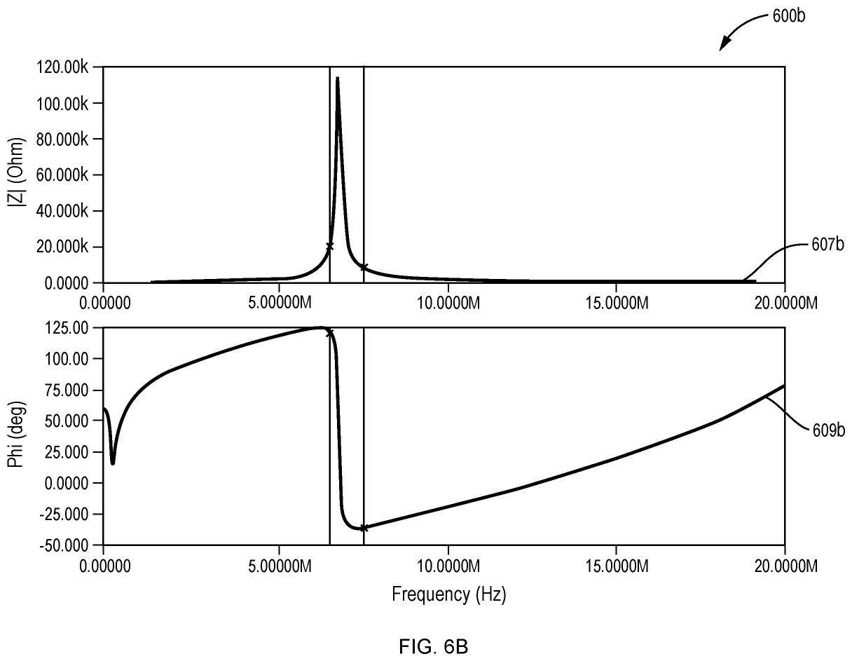

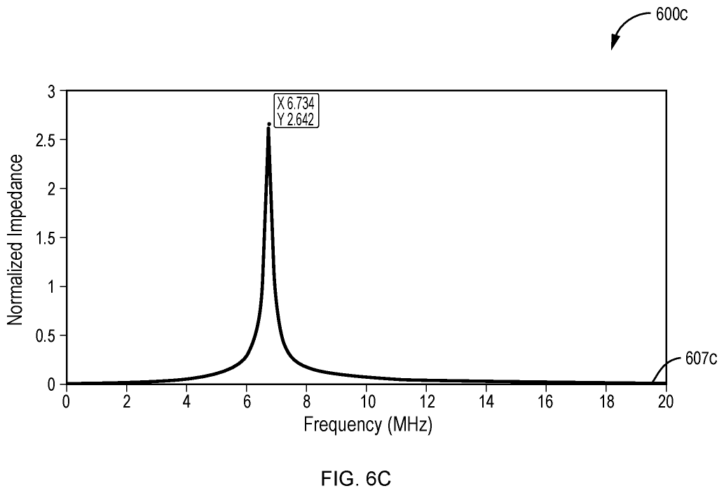

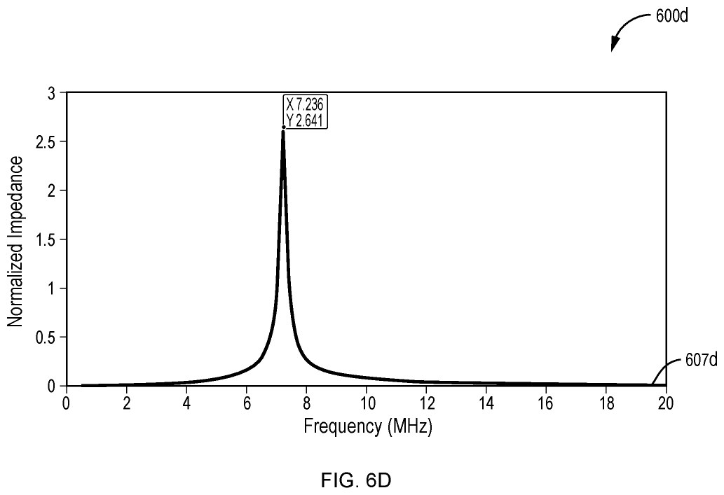

FIGS. 6A and 6B are example spectral plots 600a, 600b showing how changing number and relative location of turns of a primary coil 514 changes the resonance frequency thereof. In FIG. 6A, impedance magnitude 607a and phase 609a subplots show a resonance frequency of the primary coil 514 around 6.43 MHz before cutting. After cutting some turns and a slight change in the relative position of turns, this resonance frequency was shifted to the right as can be seen by the impedance magnitude 607b and phase 609b subplots of FIG. 6B.

FIGS. 6C and 6D are example spectral plots 600c, 600d showing a similar effect of changing the number and relative location of turns of a primary coil 514, for another iteration of the experiment as described with respect to FIGS. 6A and 6B. In FIG. 6C, impedance magnitude plot 607c shows a resonance frequency of 6.734 MHz before cutting the primary coil 514, whereas in FIG. 6D, impedance magnitude plot 607d shows that the resonance frequency has shifted to 7.236 MHz after cutting some turns and forcing a slight change in the relative position of turns of primary coil 514.

### Exemplification 3

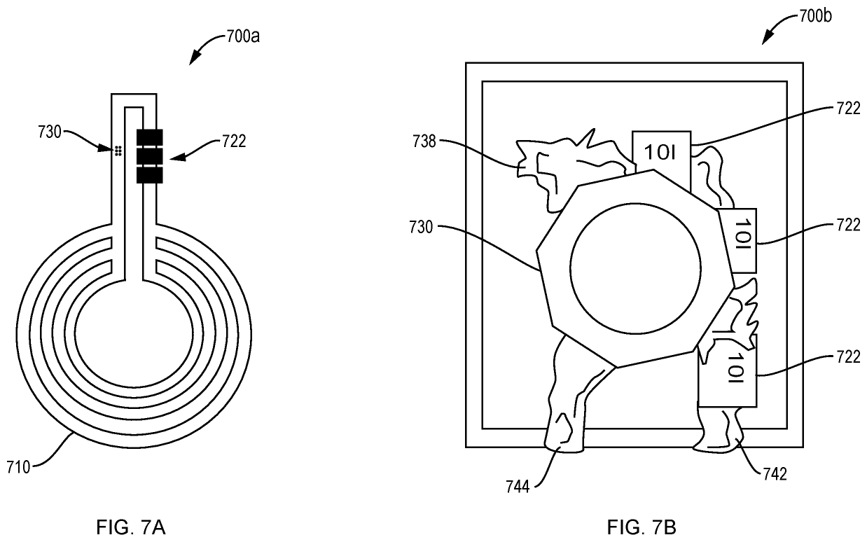

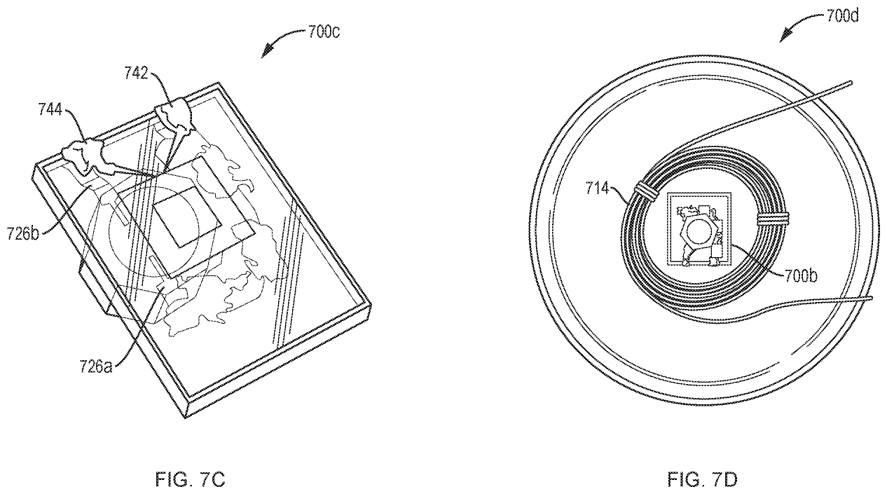

FIG. 7A is a schematic 700a of a stacked sensor embodiment of a microcoil 710. Inductors 722 and nanowell sensor 730 can be seen connected in series with microcoil 710, in a vertically stacked arrangement. In FIGS. 4A and 5, microcoils 410, 510 were physically disposed apart from nanowell sensors 430, 530; however, in FIG. 7A, microcoil 710 is part of a shared module with nanowell sensor 730.

Thus, instead of all the circuit connections associated with the secondary coil 410, 510 using wires, in some embodiments, the components 710, 714, 722 are put on the same wafer of the nanowell sensor 730 in a stacked sensor configuration. Then, one side of the inductor 722, or group thereof, may be connected to the nanowell sensor 730, and the other side to one end of the microcoil 710. The connections may be made, for example, using a conductive epoxy. The other end of the microcoil 710 may then also be connected to one side of the nanowell sensor 730.

FIG. 7B is a line drawing showing a top view 700b the stacked sensor embodiment in further detail. In FIG. 7B, the nanowell impedance sensor 730 can be seen to be connected in series with surface mount device (SMD) inductors. The other side of the nanowell sensor 730 is shown as epoxied to the microcoil 710, with the microcoil 710 located on the opposite surface. Also, the other side of the inductors 722 are shown to be epoxied to one side of the microcoil 710. Such connections between 738 the nanowell sensor 730 and inductors 722, between 742 the inductors and the microcoil 710, and between 744 the nanowell sensor 730 and the microcoil, can be seen in FIG. 7B.

FIG. 7C is a line drawing showing a bottom view 700c of the stacked sensor embodiment. Connections between 742 the microcoil 710 and inductors 722, and between 744 the microcoil 710 and nanowell sensor 730, are shown in FIG. 7C. Electrodes 726a and 726b can also be seen, electrically connected to opposing ends of the nanowell sensor 730.

FIG. 7D is a line drawing showing a combined view 700d, with the stacked sensor in a top-view orientation 700b disposed near primary coil 714. The setup of FIG. 7D was connected to a Zurich impedance spectroscope (not pictured in FIGS. 7A-D) for measurements taken to produce the results that follow.

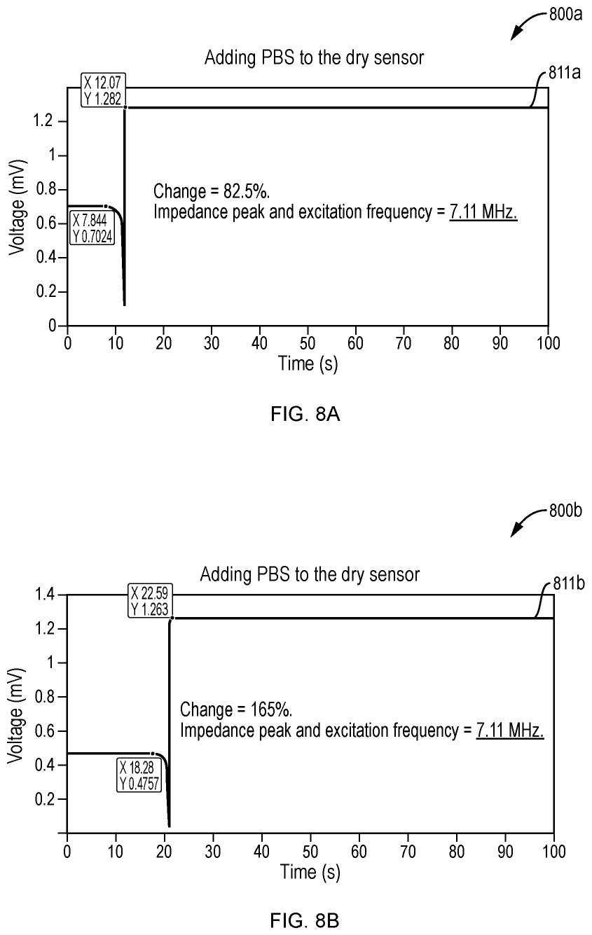

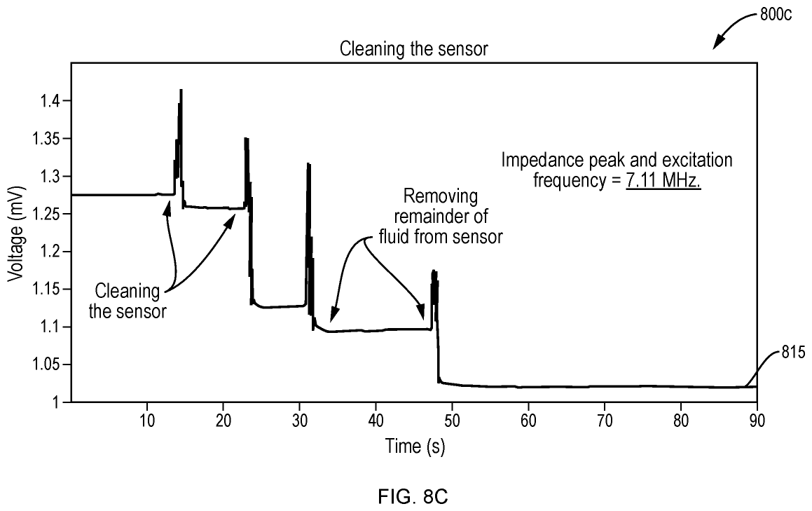

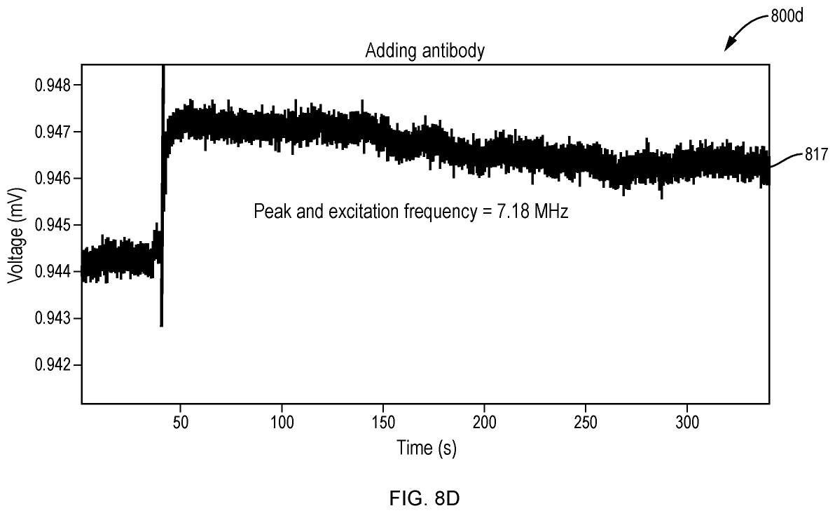

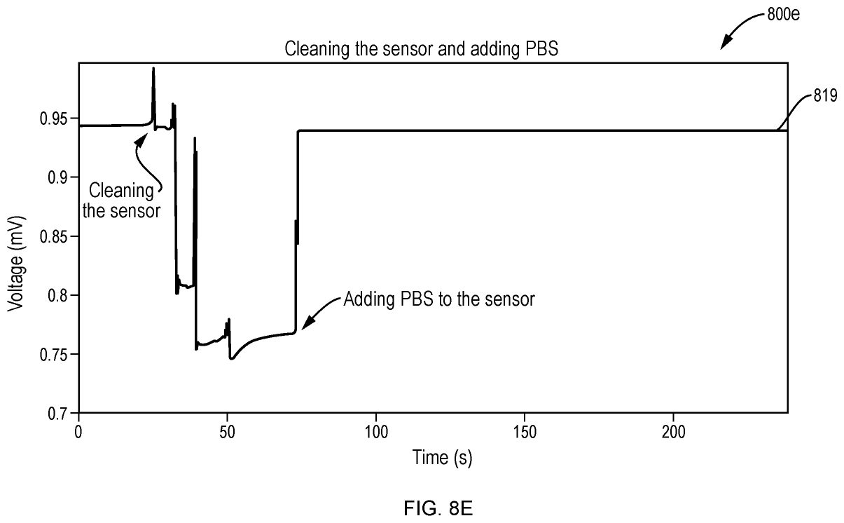

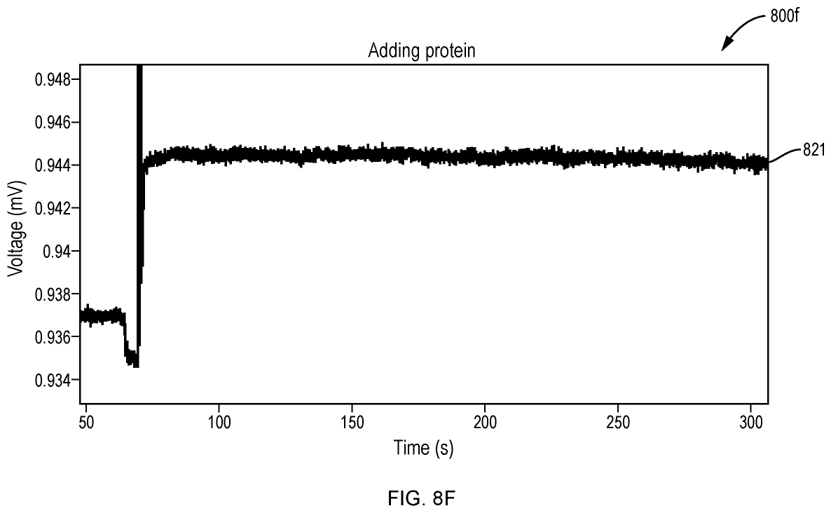

FIGS. 8A and 8B are output voltage plots 800a, 800b for two iterations of an initial experiment with the stacked sensor embodiment, wherein PBS is added to a dry sensor 730. Said two iterations used similar circuits, with differences in specific modulating components and connections thereof. Both of the resonance frequencies for the two iterations are quite close, as expected. Additionally, both iterations showed high sensitivity, as output voltage 811a, 811b can be seen to increase significantly upon addition of PBS. FIG. 8C is an output voltage plot 800c showing results 815 after removing the PBS buffer from the stacked sensor. FIG. 8D is an output voltage plot 800d showing results 817 after removing the PBS buffer and adding an antibody to the stacked sensor. After an instantaneous change, a gradual decrease was observed over time in FIG. 8D. FIG. 8E is an output voltage plot 800e showing results 819 after cleaning the stacked sensor and adding PBS to the well. A decrease in sensor output voltage was observed due to removal of the buffer and a subsequent increase was observed because of addition of PBS to the PDMS well. FIG. 8F is an output voltage plot 800f showing results 821 while adding a target protein to the sensor 730.

In some embodiments, two or more stacked sensors may be placed on top of a primary coil 714. In an example setup, the resonance frequency of primary coil 714 was closer to one of the stacked sensors which had a resonance frequency of 5.4 MHz (sensor 1). The relative location of primary coil 714 with respect to each microcoil 710 was the same. This confirmed that any difference in the output voltage change was not dependent on the relative location of primary 714 and secondary 710 coils.

### Exemplification 4

Referring back to FIG. 1A, there are many design considerations in cases of primary coil 114 design, including those described in the paragraphs that follow. In order to have the desired condition for resonance inductive coupling, the resonance frequencies of primary and secondary circuits should ideally be the same. The primary coil 114 may be the bigger external coil, which is connected to the measurement tool 194. The secondary circuit includes the nanowell impedance sensor 130 and also the secondary coil 110.

The primary coil 114 may be similar to a solenoid. So, the inductance increases while the number of turns increases, and resonance frequency becomes lower, as the resonance frequency is proportional to the reciprocal of the square root of the product of inductance and capacitance.

About the relative location of the turns, when the winding of a coil is more compact (turns are closer to one another and l is smaller), the inductance is higher and consequently resonance frequency is lower.

When the diameter of a coil is larger, the resonance frequency is lower. Diameter of respective coils has an effect on the mutual inductance of the primary and secondary coils. It can be helpful to keep the diameter at a constant value during experiments.

By including a number of extra turns in the primary coil 114, a resonance frequency appears in a lower frequency range (to the left). By cutting the turns, this frequency may be shifted higher (to the right) until it overlaps with a frequency range of the secondary coil 110.

Likewise, there are many design considerations in cases of secondary circuit design. To change the resonance frequency of the secondary circuit, inductors can be added to the secondary circuit in series with the secondary coil 110. By adding inductors 122 with the higher inductances, the resonance frequency of the secondary circuit shifts lower (to the left).

A challenge in this respect is that the nominal values of the inductors 122 are specified for specific range of frequency. These inductors 122 have a self-resonance frequency. By getting an impedance spectrum of the inductors 122, it can be seen that below this self-resonance frequency, the phase of the impedance is positive (the impedance is inductive) and that above the self-resonance frequency the phase is negative (the impedance is capacitive). Since it is necessary to add inductors to shift the resonance frequency to the left, only the inductors with a self-resonance frequency higher than the desired range of measurement frequency should be used. For example, the nominal value of a 3.3 mH inductor for the frequency of 100 kHz could be about 910 uH, and this inductor could have the self-resonance frequency of 2.5 MHz. This means that only below 2.5 MHz is this component inductive (works as an inductor).

Among SMD inductors, for example, inductors with values of 100 uH and 330 uH may have self-resonance frequencies of more than 12 MHz. These inductors may be small in size, a preferred condition for implantation.

Both spiral and square shaped coils may be used. Although some coil designs include complicated and lengthy fabrication processes, a benefit of the microcoil design employed in the setup is that it only requires a single metal layer during microfabrication.

It should be considered that even by measuring a resonance frequency of primary and secondary circuits individually, due to having mutual inductance between the coils and inductors, the resonance frequency might change when the primary 114 and secondary 110 coils are inductively coupled. So, in stacked sensor embodiments, the stacked sensor should be kept on top of the primary coil 714. For simplicity and clarity of results in experiments, only one parameter should be changed at a time. For example, parameters involved in primary resonance frequency such as number of turns, when changed, cause the two resonances to overlap, allowing protein detection measurements to be started.

To better understand how to stay in the frequency range when making design changes, simulations may be performed in simulators such as Ltspice, Simulink, Ansys and COMSOL. These simulations may provide several pieces of useful information such as aspects of the magnetic field (when the current goes through the primary coil 114 and generates a magnetic field near the secondary coil 110), and the effect of different numbers of turns and coil diameter on inductance and resonance frequencies. Also, simulations may include a nanowell sensor lumped model to obtain the required value for an inductor 122 that needs to be in series with the nanowell sensor 130 and the secondary coil 110. This value is dependent on the self-resonance frequency of available components. Moreover, the effect of the mutual inductances could change the resonance frequencies, and may be predicted by simulations.

An example for resonance frequencies of two stacked sensors could be that with multiple inductors (100 uH) were in series with the microcoil 710 and nanowell sensor 730, the resonance frequency of the secondary coils of the two sensors is measured individually (moved away from the primary coil 714 so as not to be on top of the primary coil 714). For the number of inductors 422 chosen as four inductors and six inductors, the resonance frequency was 7.38 MHz and 5.65 MHz respectively. To use these sensors in the setup, they should be put on top of the primary coil 714 and the resonance frequency of the primary coil 714 should be adjusted by changing the number of turns of the primary coil 714.

In addition to the adjustments to number of turns of a primary coil 714 described hereinabove, additional adjustments may be demonstrated as follows.

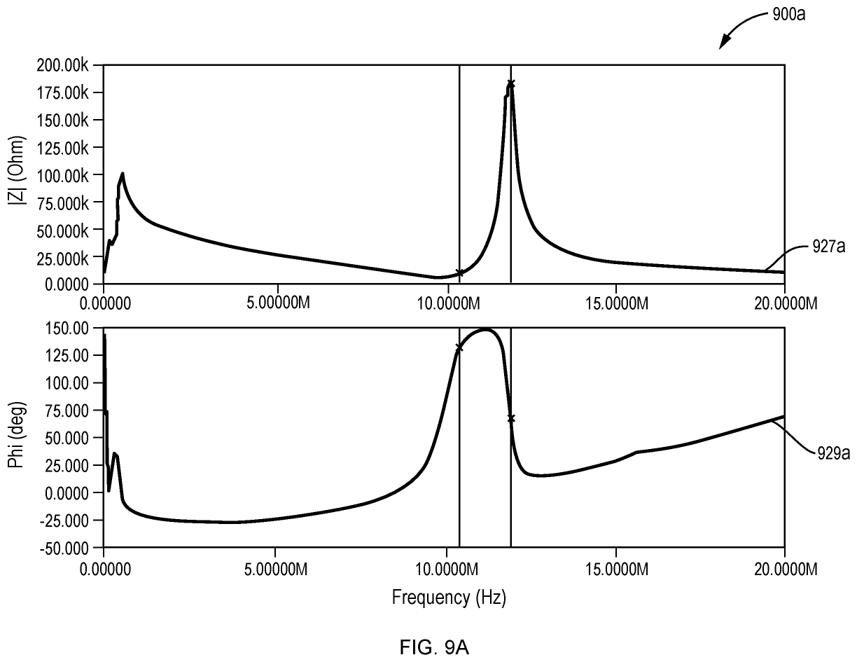

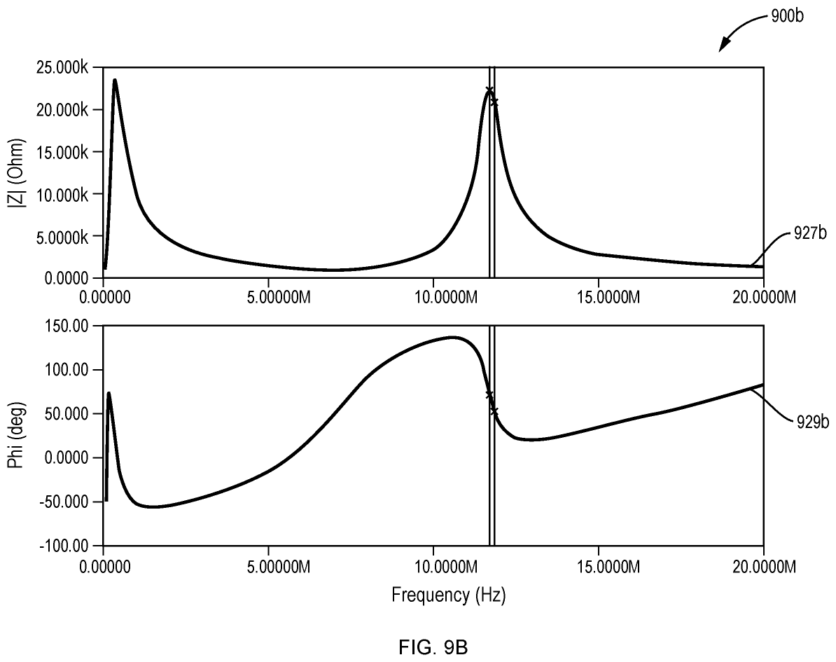

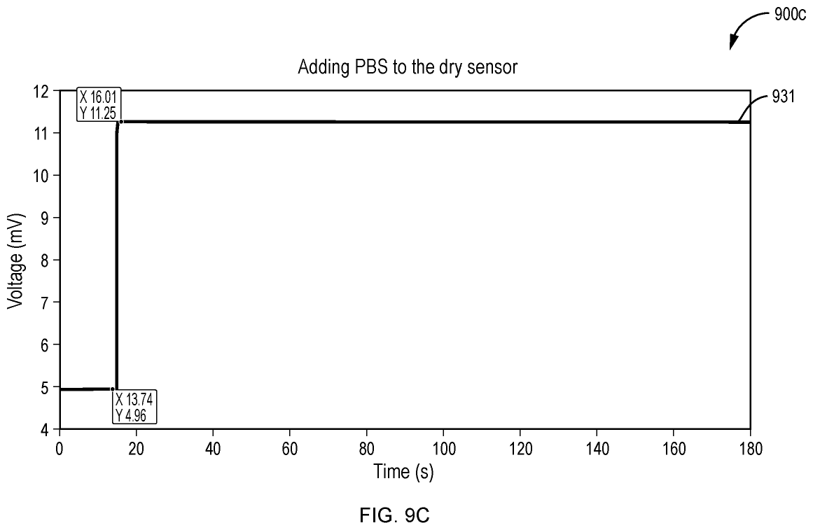

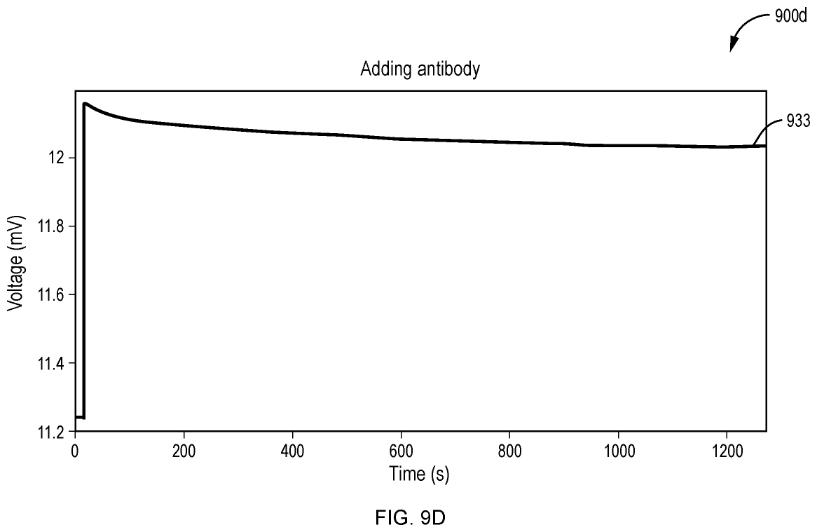

FIG. 9A is an example spectral plot 900a showing impedance magnitude 927a and phase 929a for a secondary coil 110 in cases in which resonance frequencies of primary and secondary circuits are overlapping. FIG. 9B is a counterpart example spectral plot 900b to plot 900a showing impedance magnitude 927b and phase 929b for a primary coil 114 in cases in which resonance frequencies of primary and secondary circuits are overlapping. FIG. 9C is an example output voltage plot 900c showing results 931 of adding PBS to a dry sensor in a case in which resonance frequencies of primary and secondary circuits are overlapping, featuring a notable change in output voltage. FIG. 9D is an example output voltage plot 900d showing results 933 of adding antibodies to the sensor in which resonance frequencies of primary and secondary circuits are overlapping, featuring a notable gradual decrease in output voltage after an initial instantaneous change upon adding the antibodies.

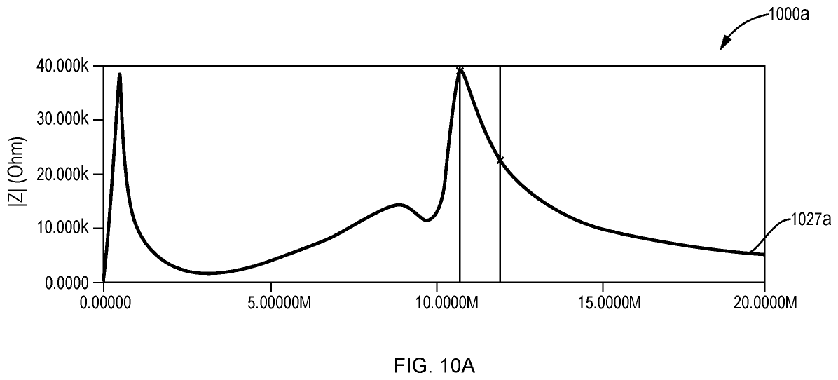

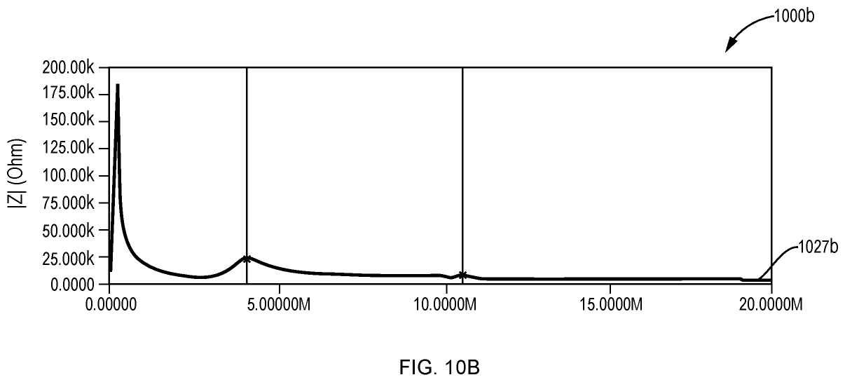

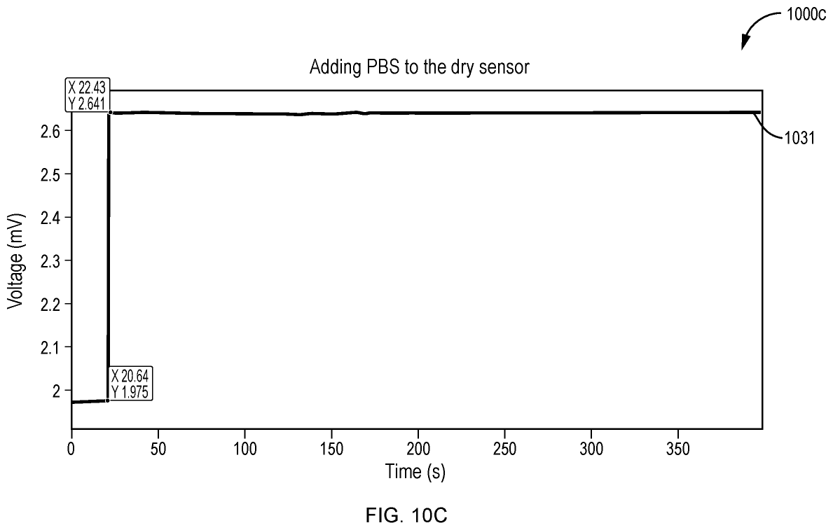

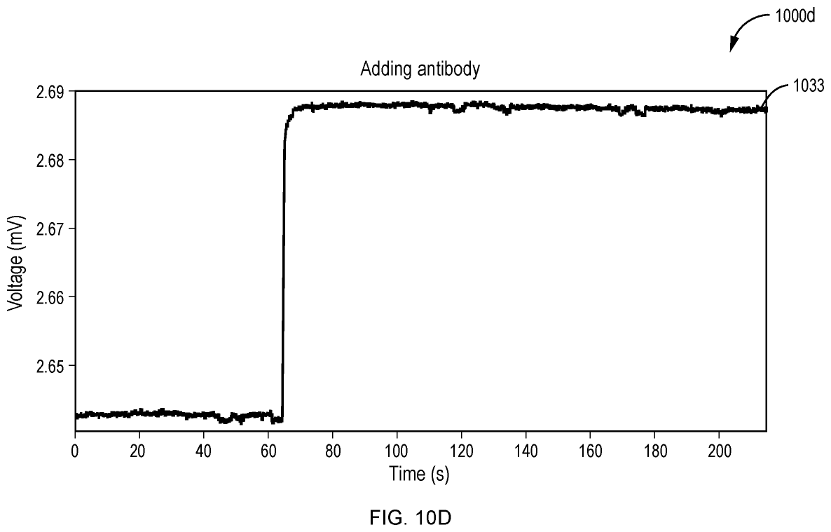

FIG. 10A is an example spectral plot 1000a showing impedance magnitude 1027a for a secondary coil 110 in cases in which resonance frequencies of primary and secondary circuits are not overlapping. FIG. 10B is a counterpart example spectral plot 1000b to plot 1000a showing impedance magnitude 1027b for a primary coil 114 in cases in which resonance frequencies of primary and secondary circuits are not overlapping. FIG. 9C is an example output voltage plot 1000c showing results 1031 of adding PBS to a dry sensor in a case in which resonance frequencies of primary and secondary circuits are not overlapping, featuring a notable change in output voltage. FIG. 10D is an example output voltage plot 1000d showing results 1033 of adding antibodies to the sensor in which resonance frequencies of primary and secondary circuits are not overlapping, showing an initial instantaneous change upon adding the antibodies, followed by a relatively stable value. In comparison with FIG. 9D, FIG. 10D shows that when the primary and secondary resonance frequencies do not overlap, the response due to the addition of the antibody is smaller than it is when the primary and secondary resonance frequencies do overlap.

Other than parameters relevant to the primary 114 and secondary 110 coils, which are important in obtaining a resonance frequency, the effect of mutual inductance should also be considered. In the following described experiments, the effect of mutual inductance on the impedance spectrum was considered.

There are two ways to measure the resonance frequency of primary and secondary circuits. One way is to build the primary and secondary circuits independently and then to connect them to an impedance analyzer or impedance spectroscope (e.g., Zurich) to measure the impedance spectrum. However, in this way the mutual inductance between the two coils is not considered while measuring the resonance frequency.

In the stacked sensor specifically other than this mutual inductance, there are other mutual inductances between the primary and secondary coils, and also between the coils and the SMD inductors added to the circuit. Therefore, a preferred embodiment includes the secondary circuit (stacked sensor) placed on top of the primary circuit.

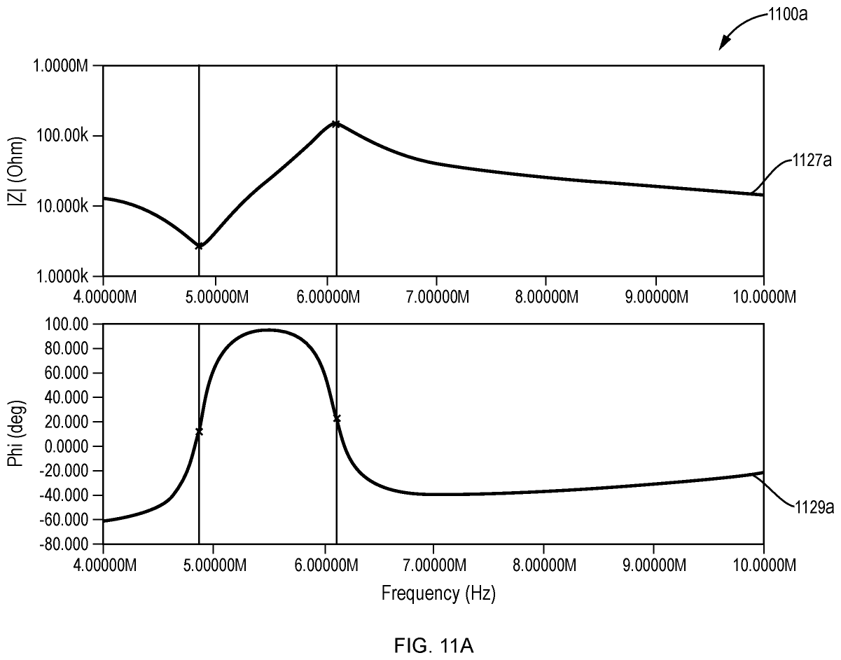

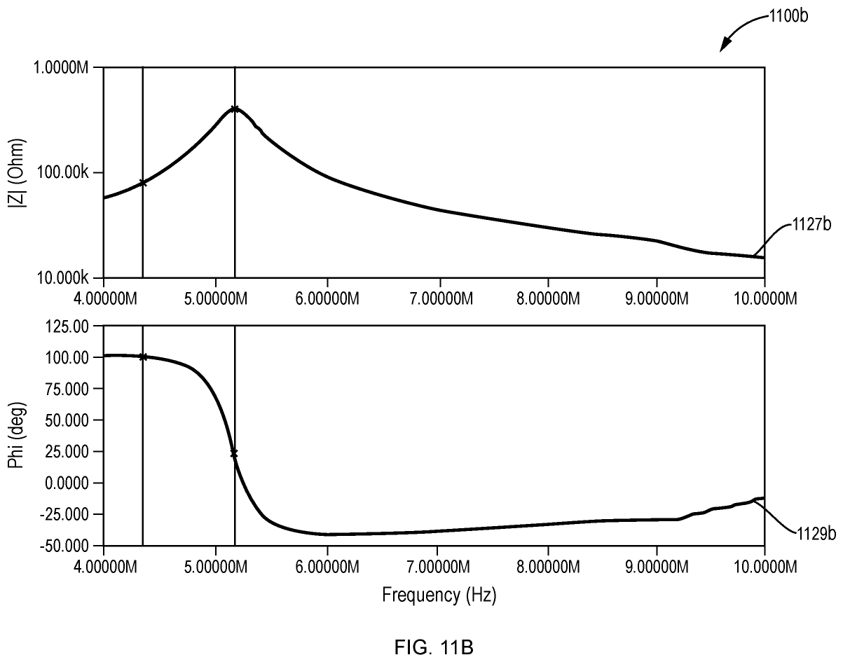

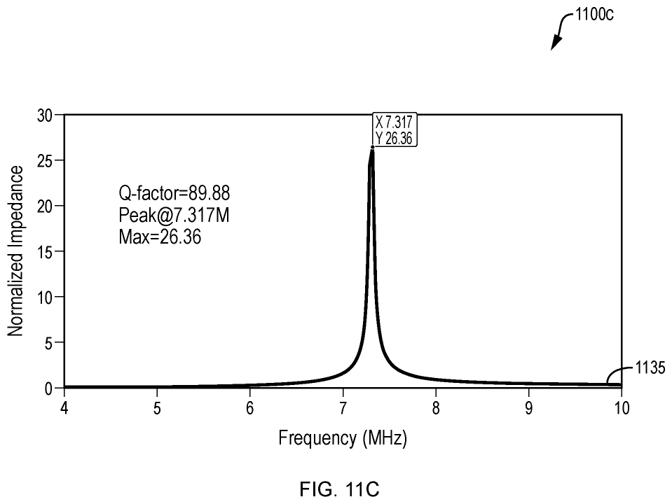

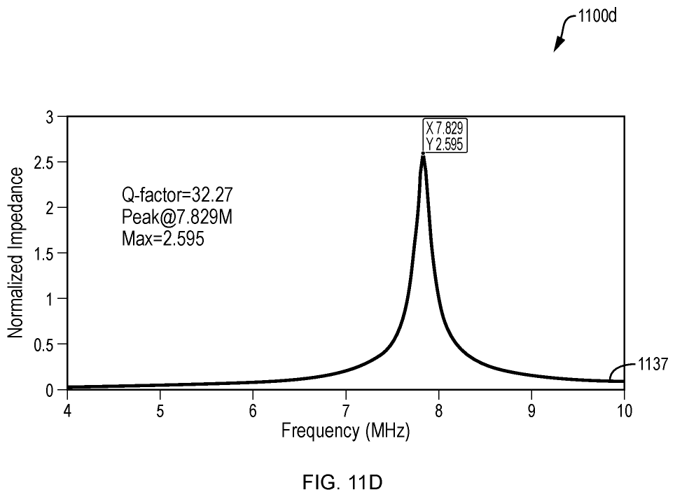

FIG. 11A is an example spectral plot 1100a showing impedance magnitude 1127a and phase 1129a for a secondary coil 110 in a stacked sensor in which the spectrum of the stacked sensor is measured individually. FIG. 11B is a spectral plot 1100b showing impedance magnitude 1127a and phase 1129a for a secondary coil 110 in a stacked sensor in which the spectrum is measured with the stacked sensor atop the primary coil 114. A peak seen in impedance magnitude 1127a for the individual measurement can be seen to have shifted down (to the left) upon measurement 1127b with the stacked sensor disposed atop the secondary coil.

FIGS. 11C and 11D are example spectral plots 1100c, 1100d showing results 1135, 1137 of shifting a resonance frequency through use of a dedicated potentiometer employed within a primary circuit. Results 1135, 1137 show that the resonance frequency does indeed shift upon operation of the potentiometer.

### Exemplification 5

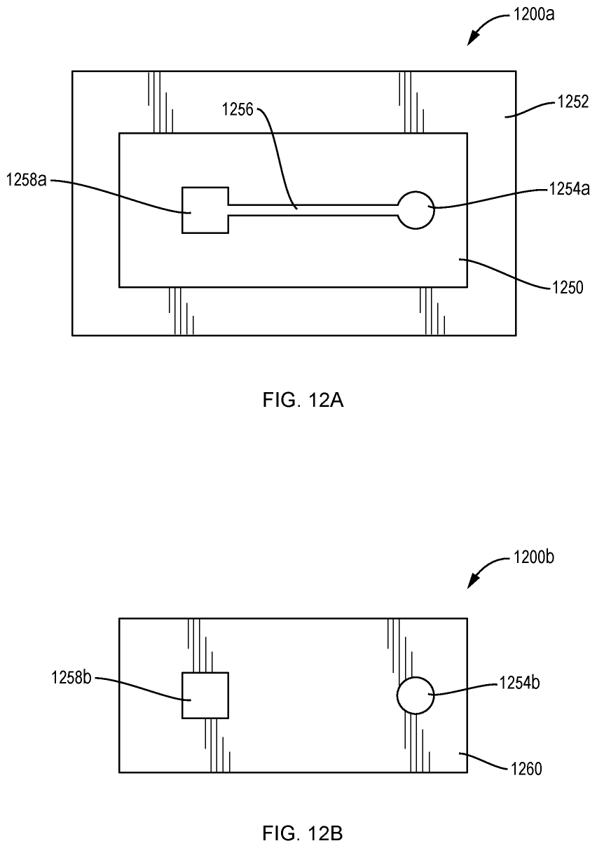

FIGS. 12A and 12B are line drawings showing components of a microfluidic test bed according to an example embodiment. Such a test bed may be used as a microfluidic wound phantom to simulate in-vivo studies.

FIG. 12A shows a view 1200a of a middle 1250 and bottom 1252 layer of such a test bed. In the figure, plural layers of double-sided tape have been patterned by a laser cutter to form a channel with walls on the middle layer 1250. The double-sided tape is placed upon a plastic substrate, which plastic substrate forms the bottom layer 1252. Other substrate materials may be used, such as glass. The middle layer 1250 forms an inlet 1254a and an outlet 1258a at opposing ends of the channel.

FIG. 12B shows a view 1200b of a top layer 1260 of such a test bed, formed from plastic. Cut-outs for the inlet 1254b and outlet 1258b are shown, corresponding to inlet 1254a and outlet 1254a of the middle layer 1250. The top layer 1260 is thus arranged and attached atop the middle layer 1250 with inlets 1254a, 1254b and outlets 1258a, 1258b substantially aligned. A nanowell sensor such as nanowell sensor 130 may be fitted and attached to/inserted into the outlet. As such, the outlet may function to simulate a wound into which a nanowell sensor 130 may be inserted to simulate an in-vivo biosensing implementation.

### Exemplification 6

In a pair of experiments, a nanowell sensor was exposed to cell cultures, and an animal's wound, over a period of time. Such biocompatibility experiments had two primary goals. The first goal was to observe how the sensor affects an animal or a growth medium over time. The second goal was to observe whether the biological environment affected the sensor's performance.

In a first experiment, a nanowell sensor was placed inside a cell growth medium and the medium was monitored to determine whether there was any change in the cell growth medium caused by the sensor. In a second experiment, a nanowell sensor was placed on a mouse's wound to monitor its change. It was observed that placing the sensor inside the cell growth medium did not change the morphology or number of cells. Also, the sensors were functional after biocompatibility experiments. It can be seen that wound closure happens at the same time for both the control and test groups in biocompatibility tests with the animal. Finally, none of the sensors were shorted after the experiment.

MDA-MB-468 breast cancer cells were used in the first set of biocompatibility experiments. In this respect, 10 plates were used to grow cancer cells. The initial number of cells in each plate was 5000. However, the number of cells increases over the time. During the first 24 hours, no sensor was added to any container (plate) to make sure that all containers have the same condition until the 24-hour time point. Then, at 24 hours, the plates were divided into control and test groups. In this respect, one sensor was placed in each of the five containers. The other containers did not have any sensors, forming a control group.

After an additional 24 hours, the number of live vs. dead cells was compared for the control group and the test group. So, the sensor was removed from one of the plates in the test group, and the number of cells therein was compared with the number of cells in one of the plates in control group. This process was continued for the next days of the experiment.

For counting the cells at different time points, vials containing cells were prepared in each plate. In this respect, dissociation solution was added to both plates (one from the control group and another from the test group). This makes the cells come off the surface and become suspended in the solution. Then, the plates were put in an incubator for 10 minutes. Then, put the solution was added to the vial. Finally, growth medium was added to the plate to wash the remainder of the cells, and this remainder was also added to the vial. In a final step, the vials were put in flow cytometry holders to start counting the viable cells.

Morphology of the cells in two groups was checked at different time points to check if there was any change due to the sensor in the cancer cell growth medium. Results indicated that there was no change in morphology/shape in presence of the sensor device for 7 days. The cells were grown in 3D, so, in the test group, it had been expected that a difference would be seen between the morphology of the cells under the sensor and other areas in the same plate even before starting the experiment. The results indicated that there was no change in morphology/shape in presence of the sensor device. Besides checking the morphology of the cells, the number of cells in both plates was enumerated for different intervals of days.

After taking the sensors out of the growth medium, they were cleaned, and PBS was added to the sensor wells to see if they were still working. Some of the sensors were in good working condition. However, some of them were shorted or they were not in the desirable condition. In another biocompatibility experiment with cell culture medium, this problem was addressed by replacing the conductive epoxy.

For the second experiment, biocompatibility tests were performed with a nanowell sensor placed upon a mouse's wound. The PDMS well used previously had a height of about a few millimeters. However, a thinner layer of PDMS was needed on the sensor in order to be placed on the animal skin. So, liquid PDMS was cast upon a silicon wafer. Then, different speeds of a spin-coater were used.

Sharp corners were eliminated from singulated nanowell sensors because they may lead to inflammation. Different methods were employed to smooth the corners. First, using a laser cutter, curved shapes were created on the corners. The corners were much more rounded and were not sharp anymore after using laser cutter for the corners. However, the sensors were then observed to be shorted. This might be due to different steps for making connections and cutting edges involved in heating up the sensor. Finally, sand paper was used for the corners and the edges. Also, a very thin layer of epoxy was used on the corner.

The experiment was conducted as follows. There is a sensor in contact with one wound on a mouse, and another wound is the control. At time point zero, the sensor was placed within the wound. The sensor was removed from the wound at 24 hours and tested with PBS to see if it was still working.

Then, photos were taken at the next time points (48 hours, 72 hours, and 96 hours) from the control and test wound of this animal. In this way, by comparing wound windows, wound inflammation in both groups can be compared. Also, after the sensor was taken out of the wound, wound fluid samples from the two wounds were taken. These samples were frozen at −30° C. On the final day of the experiment, the wound fluid samples from the two groups (test and control), were tested with enzyme-linked immunosorbent assay (ELISA) kits to measure IL-6 or TNF-α as inflammatory markers.

Additional biocompatibility experiments were performed with a subsequent group of animals. In these experiments 10 mice were used. Three of the animals were used as controls. A sensor was placed on a wound of each of the other animals and removed from the wounds at specific timepoints. The wound size was monitored to study the rate at which the wound is healed and wound closure happens.

Also, wound fluid samples were collected from all 10 animals at different timepoints on day 0, day 2, day 4, day 6, day 8, and finally day 10, and these samples were used to test for cytokines. These samples were then frozen in −30° C. and were used at the time of performing ELISA. The ELISA experiments showed that there was no significant difference between the level of cytokines (IL-6 and TNF-α) between the control and test groups. For example, to measure the difference between the two groups of animals (i.e., the control group and test group), 2-way ANOVA was performed. The results indicated that the p-value was greater than 0.05, which indicates that differences between groups are not significant at different time points (e.g., at day 0, 2, 4, 6, 8, and 10).

It was observed that placing the sensor inside the cell growth medium did not change the morphology/number of cells. Also, the sensors were functional after biocompatibility experiments. We also saw that wound closure happens at the same time for both control and test groups in biocompatibility tests with animals. Also, none of the sensors were shorted after the experiment.

### Exemplification 7

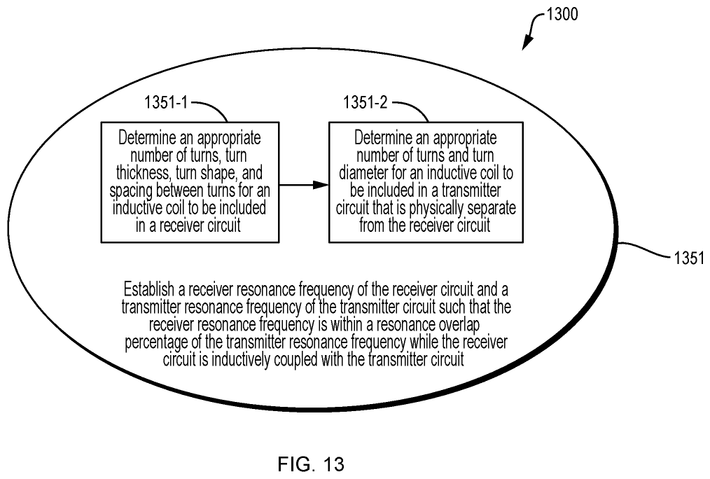

FIG. 13 is a diagram depicting an example embodiment of a method 1300 of manufacturing a measurement system for detecting a target analyte in a sample, such as, for example, the system 100. The method 1300 includes determining 1351-1 an appropriate number of turns, turn thickness, turn shape, and spacing between turns for an inductive coil to be included in a receiver circuit 110. The method 1300 further includes determining 1352-2 an appropriate number of turns and turn diameter for an inductive coil to be included in a transmitter circuit 114 that is physically separate from the receiver circuit, to establish 1351 a receiver resonance frequency of the receiver circuit and a transmitter resonance frequency of the transmitter circuit such that the receiver resonance frequency is within a resonance overlap percentage of the transmitter resonance frequency while the receiver circuit is inductively coupled with the transmitter circuit.

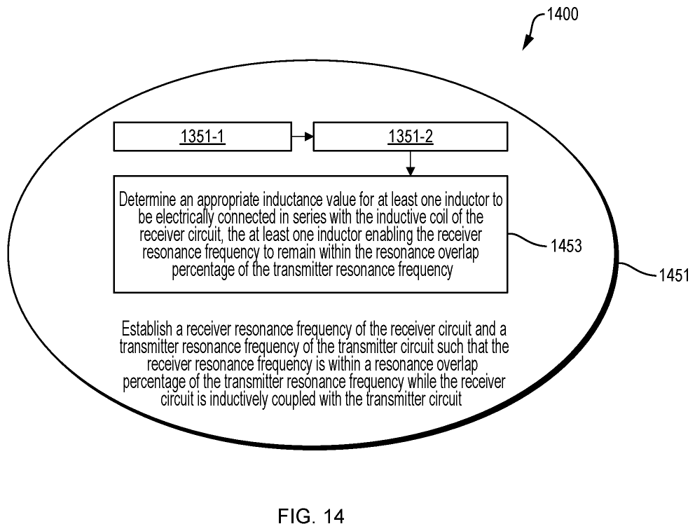

FIG. 14 is a diagram depicting an example embodiment of a method 1400 of manufacturing a measurement system for detecting a target analyte in a sample, such as, for example, the system 100. The method 1400 includes steps 1351-1 and 1352-2 of method 1300. The method 1400 further includes determining 1453 an appropriate inductance value for at least one inductor 122 to be electrically connected in series with the inductive coil of the receiver circuit 110, the at least one inductor 122 enabling the receiver resonance frequency to remain within the resonance overlap percentage of the transmitter resonance frequency. The method 1400 thereby establishes 1451 a receiver resonance frequency of the receiver circuit and a transmitter resonance frequency of the transmitter circuit such that the receiver resonance frequency is within a resonance overlap percentage of the transmitter resonance frequency while the receiver circuit is inductively coupled with the transmitter circuit. In one example, the resonance overlap percentage is 10% and the signal overlap percentage is at least 60%.

The methods 1300, 1400 can include selecting a turn shape and values for number of turns, turn thickness, and spacing between turns for the inductive coil of the receiver circuit 110 to establish the receiver resonance frequency within a range of 4 to 50 MHz. The methods 1300, 1400 can further include selecting a number of turns and turn diameter for the inductive coil of the transmitter circuit 114 to establish the transmitter resonance frequency within a range of 4 to 50 MHz.

The methods 1300, 1400 can include lithographically fabricating the inductive coil of the receiver circuit 110 as a planar coil disposed upon an implantable substrate. The methods 1300, 1400 can include electrically connecting a lithographically fabricated inductive coil of the receiver circuit 110 to electrodes 126a, 126b of a nanowell sensor 130 that is (i) co-fabricated with the inductive coil 110 upon an implantable substrate, wherein the implantable substrate, after singulation from a parent wafer, has no dimension greater than one centimeter so as to facilitate implantation within a human or animal body or a part thereof; or (ii) physically mated with the implantable substrate 130 within an implantable module, wherein the implantable module has no dimension greater than one centimeter so as to facilitate implantation within a human or animal body or a part thereof.

### Exemplification 8

FIG. 15 schematic diagram 1500 illustrating an example embodiment, wherein the transmitter circuit includes a variable resistor 1576, a primary coil 1514, and a lock-in-amplifier 1594, e.g. a Zurich impedance spectroscope (Zurich Instruments HF2IS, Zurich, SI). The lock-in amplifier 1594 measures the complex impedance in real-time to detect binding events involving proteins or other biomarkers in the nanowell sensor 1530. The primary coil 1514 is inductively coupled with a secondary coil 1510 connected in series with the nanowell sensor 1530 and a variable resistor 1524 in the receiver circuit. The secondary circuit also includes an inductor 1522. In one example, the inductor 1522 is a 270 uH inductor.

In the aforementioned example embodiment, the resonance frequency of the primary 1514 and secondary 1510 coils are different, due to a difference in number of turns and size of the coil. The mismatch of the resonance frequency reduces the sensitivity of the sensor 1530. Tuning the variable components 1524, 1576 on the primary and/or secondary side results in modulating the resonance frequency of the same side, and a better match to the other side.

In this setup, resonant frequency is depending on the value of variable resistor 1524, 1576. In an example, the variable resistor ranges from 100 kΩ to 10 kΩ, and results in the resonant frequency being tuned from 6.8 MHz to 9 MHz.

The variable component on both primary and secondary sides may be the variable resistor 1524 1576, whose range may be, for example, 100 Ω to 10 kΩ. The variable component on the primary side 1524 modulates the resonance frequency of the primary side from, for example, 5.982 MHz to 9.210 MHz. The lower boundary of the resonance frequency, 5.982 MHz, is corresponding to scenarios when the component is set to 100Ω, while the upper boundary, 9.210 MHz, occurs when the variable component set to 10 kΩ. The variable component on the secondary side 1524 modulates the resonance frequency of receiver loop, including the nanowell sensor 1530, from 6.543 MHz to 9.004 MHz. The lower boundary of the resonance frequency, 6.543 MHz, is corresponding to when the variable component 1524 was set to 100Ω, while the upper boundary, 9.004 MHZ, occurs when the variable component set to 10 kΩ. On the secondary side, a 270 uH inductor was used, consisting of the frequency modulation component 1525, and significantly increases the circuit response of sensor 1530. The optimized excitation frequency is 6.82 MHz, which matches the resonance frequency of both primary and secondary sides. This configuration happens when the variable component of the primary side 1524 is set to about 1 kΩ, and the secondary's set to 100Ω.

### Exemplification 9

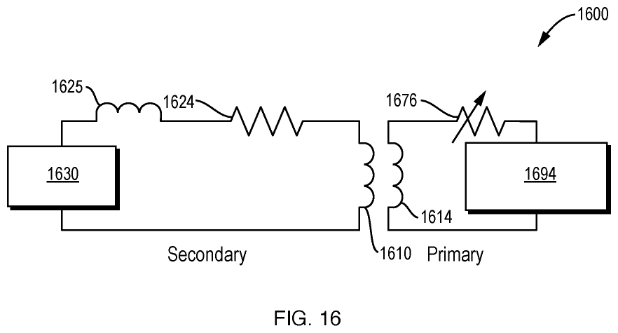

FIG. 16 schematic diagram 1600 illustrating an example embodiment, wherein the transmitter circuit includes a variable resistor 1676, a primary coil 1614, and a lock-in-amplifier 1694, e.g. a Zurich impedance spectroscope (Zurich Instruments HF2IS, Zurich, SI). The lock-in amplifier 1694 measures the complex impedance in real-time to detect binding events involving proteins or other biomarkers in the nanowell sensor 1630. The primary coil 1614 is inductively coupled with a secondary coil 1610 connected in series with the nanowell sensor 1630 and a resistor 1624 in the receiver circuit. The secondary circuit also includes an inductor 1622. In one example, the inductor 1622 is a 270 μH inductor and the resistor 1624 is a 100Ω resistor.

The example setup shown in FIG. 16 may be used in a modified stacked sensor embodiment, such as a modified version of the embodiment of FIGS. 7A-D. The sensor 1630 may be configured to mate with a microfluidic cell such as is shown in FIGS. 12A and 12B, which may in turn be in contact with a wound or a wound phantom. One terminal of the secondary coil 1610 may be connected to one terminal of resistor 1624, e.g., with epoxy. The other terminal of resistor 1624 may be connected to the nanowell sensor 1630. The second terminal of the secondary coil 1610 may be connected to an inductor 1625 or network thereof. The inductor 1625 or network thereof, may in turn be connected, at its other end, to the opposite side of the nanowell sensor 1630 from the secondary coil 1610, forming a closed loop.

The variable component on the primary side is the variable resistor 1676, whose range may be 100Ω to 10 kΩ. The variable component on the primary side 1676 modulates the resonance frequency of the primary side, for example, from 5.982 MHz to 9.210 MHz. The lower boundary of the resonance frequency 5.982 MHz is corresponding to scenarios when the component 1676 is set to 100 $2, while the upper boundary 9.210 MHz occurs when the variable component set to 10 kΩ. The resonance frequency of the secondary side may be optimized at 6.8 MHz, using a combination of 100Ω resistor 1624 and 270 uH inductor 1625. The receiver coil 1610, frequency modulation circuit, and the nanowell sensor 1630 was built and connected on stacked chips.

### Exemplification 10

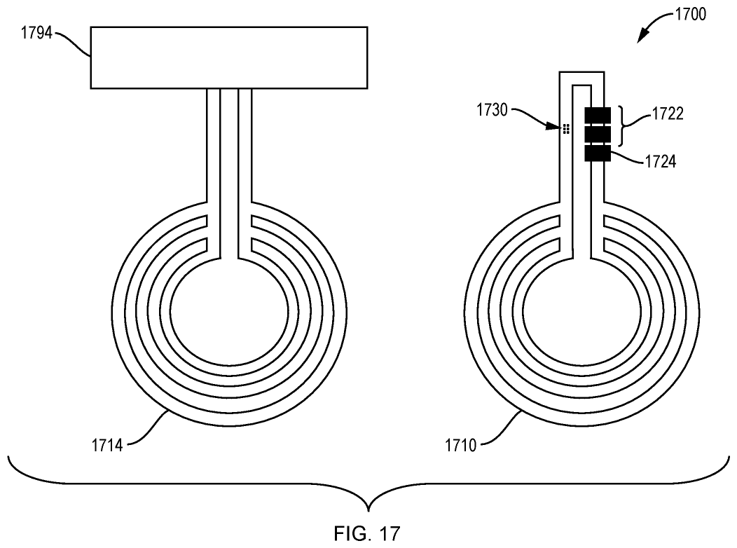

FIG. 17 is a schematic diagram 1700 illustrating an example embodiment, in which commercially available RFID coils are used to design a wireless setup to modulate protein binding to the measured impedance. In this regard, commercially available RFID coils were used as the transmitter coil 1714 and as the receiver coil 1710. After measurement of the resonance frequency of the transmitter circuit, components such as surface-mount device (SMD) inductors 1722 and/or resistors 1724 were used to make the resonance frequency of the transmitter and receiver circuits overlap. This resonance frequency may be, for example, 24.48 MHz.

RFID coils 1710, 1714 may be prepared by scratching the cover under a microscope and connecting the two ends to the circuit using epoxy. Both primary 1714 and secondary 1710 in this setup are RFID coils. The secondary coil 1710 is placed on top of the primary coil 1714 while it is in series with the nanowell sensor 1730 and modulation components 1722, 1724. Components 1722, 1724 are used to make resonance frequencies of primary 1714 and secondary 1710 overlap. Primary coil 1710 is connected to a lock-in amplifier measurement system 1794 such as a Zurich impedance spectroscope.

### REFERENCES

[1] Kim, Han-Joon, et al. “Review of near-field wireless power and communication for biomedical applications.” IEEE Access 5 (2017): 21264-21285.
[2] Xie, Pengfei, et al. “A ten-minute, single step, label-free, sample-to-answer assay for qualitative detection of cytokines in serum at femtomolar levels.” Biomedical Microdevices 22.4 (2020): 1-9.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A device for detecting a target analyte in a sample, the device comprising:
a nanowell impedance sensor configured to receive a sample including a target analyte, an impedance between electrodes of the sensor being modulated by the target analyte when the target analyte is present in the sample, the modulated impedance being indicative of a concentration of the target analyte in the sample; and
a receiver circuit connected across the electrodes and having a receiver resonance frequency, the receiver circuit including an inductive coil configured to induce an electrical current in the receiver circuit while inductively coupled with a physically separate transmitter circuit having a transmitter resonance frequency, the receiver resonance frequency being within a resonance overlap percentage of the transmitter resonance frequency;
wherein:
the electrical current induced in the receiver circuit is a time-varying signal having a frequency within a signal overlap percentage of the receiver resonance frequency; and
the modulated impedance is determined as a function of the electrical current induced, thereby detecting the presence of the target analyte, and the concentration thereof, in the sample.

2. The device of claim 1 wherein the inductive coil of the receiver circuit:
has a number of turns between 5 and 60;
has a distance between turns of between 10 and 30 microns; and
has a turn width between 10 and 30 microns.

3. The device of claim 1 wherein the inductive coil of the receiver circuit is characterized by a square or a circular spiral geometry.

4. The device of claim 1 wherein the receiver circuit includes at least one inductor connected in series with the inductive coil of the receiver circuit, the at least one inductor enabling the receiver resonance frequency to remain within the resonance overlap percentage of the transmitter resonance frequency.

5. The device of claim 1 wherein the resonance overlap percentage is 10%, and wherein the signal overlap percentage is at least 60%.

6. The device of claim 1 wherein the receiver resonance frequency is within a range of 4 to 50 MHz.

7. The device of claim 1 wherein the device occupies an area of less than one square centimeter so as to be implantable within a human or animal body or a part thereof.

8. A measurement system for detecting a target analyte in a sample, the system comprising:
a nanowell impedance sensor configured to receive a sample including a target analyte, an impedance between electrodes of the sensor being modulated by the target analyte when the target analyte is present in the sample, the modulated impedance being indicative of a concentration of the target analyte in the sample;
a receiver circuit connected across the electrodes and having a receiver resonance frequency, the receiver circuit including an inductive coil; and
a transmitter circuit having a transmitter resonance frequency such that the receiver resonance frequency is within a resonance overlap percentage of the transmitter resonance frequency, the transmitter circuit including:
a voltage source configured to apply an electrical voltage to the inductive coil of the transmitter circuit, so as to induce the electrical current in the receiver circuit, causing an electrical voltage to be applied across the electrodes of the nanowell sensor; and
an inductive coil configured to be wirelessly coupled with the inductive coil of the receiver circuit by mutual inductance such that the receiver circuit is inductively coupled with the transmitter circuit;
wherein:
the electrical current induced in the receiver circuit is a time-varying signal having a frequency within a signal overlap percentage of the receiver resonance frequency; and
the modulated impedance is determined as a function of the electrical current induced, thereby detecting the presence of the target analyte, and the concentration thereof, in the sample.

9. The system of claim 8 wherein the transmitter circuit includes a lock-in amplifier and a measurement device configured to measure a current flowing in the transmitter circuit to enable the modulated impedance to be determined further as a function of a current measured.

10. The system of claim 8 wherein the transmitter circuit includes a measurement device configured to measure an impedance spectrum across the inductive coil of the transmitter circuit to enable the modulated impedance to be determined further as a function of a measured change in the resonance frequency of the receiver circuit in response to a change in the concentration of the target analyte present in the sample.

11. The system of claim 8 wherein the receiver circuit includes at least one inductor connected in series with the inductive coil of the receiver circuit, the at least one inductor enabling the receiver resonance frequency to remain within the resonance overlap percentage of the transmitter resonance frequency.

12. The system of claim 8 wherein the resonance overlap percentage is 10%, and wherein the signal overlap percentage is at least 60%.

13. The system of claim 8 wherein the receiver resonance frequency and the transmitter resonance frequency are within a range of 4 to 50 MHz.

14. The system of claim 8 wherein the inductive coil of the transmitter circuit has a number of turns between 10 and 200.

15. A method of manufacturing a measurement system for detecting a target analyte in a sample, the method comprising determining an appropriate number of turns, turn thickness, turn shape, and spacing between turns for an inductive coil to be included in a receiver circuit, and an appropriate number of turns and turn diameter for an inductive coil to be included in a transmitter circuit that is physically separate from the receiver circuit, to establish a receiver resonance frequency of the receiver circuit and a transmitter resonance frequency of the transmitter circuit such that the receiver resonance frequency is within a resonance overlap percentage of the transmitter resonance frequency while the receiver circuit is inductively coupled with the transmitter circuit.

16. The method of claim 15 further comprising determining an appropriate inductance value for at least one inductor to be electrically connected in series with the inductive coil of the receiver circuit, the at least one inductor enabling the receiver resonance frequency to remain within the resonance overlap percentage of the transmitter resonance frequency.

17. The method of claim 15 wherein the resonance overlap percentage is 10%, and wherein the signal overlap percentage is at least 60%.

18. The method of claim 15 further comprising:
selecting a turn shape and values for number of turns, turn thickness, and spacing between turns for the inductive coil of the receiver circuit to establish the receiver resonance frequency within a range of 4 to 50 MHz; and
selecting a number of turns and turn diameter for the inductive coil of the transmitter circuit to establish the transmitter resonance frequency within a range of 4 to 50 MHz.

19. The method of claim 15 further comprising lithographically fabricating the inductive coil of the receiver circuit as a planar coil disposed upon an implantable substrate.

20. The method of claim 19 further comprising electrically connecting the lithographically fabricated inductive coil of the receiver circuit to electrodes of a nanowell sensor that is (i) co-fabricated with the inductive coil upon the implantable substrate, wherein the implantable substrate, after singulation from a parent wafer, has no dimension greater than one centimeter so as to facilitate implantation within a human or animal body or a part thereof; or (ii) physically mated with the implantable substrate within an implantable module, wherein the implantable module has no dimension greater than one centimeter so as to facilitate implantation within a human or animal body or a part thereof.
